(19) United States
(12) Patent Application Publication
(10) Pub. No.: US 2010/0056507 A1
(43) Pub. Date: Mar. 4, 2010

(54) PHENYL DERIVATIVES AND THEIR USE AS A MEDICAMENT

(75) Inventors: Lydie Poitout, Antony (FR); Jeremiah Harnett, Gif-sur-Yvette (FR); Dennis Bigg, Gif-sur-Yvette (FR); Carole Sackur, Paris (FR); Eric Ferrandis, Saint Remy les Chevreuse (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: IPSEN PHARMA S.A.S., Boulogne-Billancourt (FR)

(21) Appl. No.: 12/447,437

(22) PCT Filed: Oct. 26, 2007
(86) PCT No.: PCT/FR2007/001776

§ 371 Date: Apr. 27, 2009

(30) Foreign Application Priority Data
Oct. 27, 2006 (FR) 0609436

(51) Int. Cl. A61K 31/536; C07C 69/76; A61K 31/235; C07D 271/10; A61K 31/4245; C07D 271/06; C07D 215/00; A61K 31/47; C07D 265/36; C07D 285/12; A61K 31/433; A61P 35/00; A61P 37/06; A61P 1/00; A61P 29/00

(52) U.S. Cl. 514/230.5; 560/100; 514/544; 548/143; 514/364; 548/131; 546/152; 514/311; 544/105; 548/136; 514/363

(57) ABSTRACT

The present invention relates to new phenylic derivatives exhibiting a good affinity for certain sub-types of cannabinoid receptors, in particular the CB2 receptors. These derivatives are of particular interest in a method for treating pathological conditions and diseases in which one or more cannabinoid receptors are involved. The invention also relates to pharmaceutical compositions containing said new phenylic derivatives and to methods for the preparation and use thereof.

A subject of the present application is novel phenyl derivatives. These products have a good affinity for certain sub-types of cannabinoid receptors, in particular the CB2 receptors. They are particularly useful for treating the pathological states and the diseases in which one or more cannabinoid receptors are involved. The invention also relates to pharmaceutical compositions containing said products and their use for the preparation of a medicament.

The cannabinoids are psychoactive components present in Indian cannabis (Cannabis sativa) including approximately 6 different molecules, the most represented of which is delta-9-tetrahydrocannabinol. Knowledge of the therapeutic activity of cannabis goes back to the ancient Chinese dynasties in which, 5000 years ago, cannabis was used for the treatment of asthma, migraines and gynaecological disorders. It was in 1850 that cannabis extracts were recognized and included in the American pharmacopoeia.

The cannabinoids are known for having different effects on numerous functions and organs, the most important being on the central nervous system and on the cardiovascular system. These effects include alterations to the memory, euphoria and sedation. The cannabinoids also increase the pulse and modify the systemic arterial pressure. Peripheral effects linked to bronchial constriction, immunomodulation and inflammation have also been observed. More recently, it has been shown that the cannabinoids modulate the cellular and humoral immune responses and possess anti-inflammatory properties. Despite all of these properties, the therapeutic use of the cannabinoids is controversial because of their psychoactive effects (cause of dependency) but also for their multiple side effects which have not yet been completely characterized. Although numerous works have been carried out in this field since the 1940s, little information existed on the characterization of cannabinoid receptors, the existence of endogenous ligands and until recently on selective products of a particular receptor sub-type.

Two cannabinoid receptors have been identified and cloned, CB1 and CB2. CB1 is expressed predominantly in the central nervous system whereas CB2 is expressed in the peripheral tissues, mainly in the immune system. These two receptors are members of the family of the receptors coupled to the G proteins and their inhibition is linked to the activity of adenylate cyclase.

On the basis of all this information, a need exists for compounds capable of selectively modulating the cannabinoid receptors and therefore the pathologies associated with such receptors. Thus, CB2 modulators offer a single pharmacotherapeutical approach against immune disorders, inflammation, osteoporosis, renal ischaemia and other pathological states. There is a considerable interest in developing cannabinoid analogues having a strong affinity for the CB2 receptor. Cannabinoid analogues which specifically modulate the CB2 receptor, directly or indirectly, can produce clinically useful effects without affecting the central nervous system, thus providing a rational therapeutic approach for a wide variety of pathological states.

The novel compounds of this invention modulate the activity of CB2 and are as a result useful for the treatment and prevention of the pathological states and diseases associated with the activity of the cannabinoid receptors such as, but not limited to, cell proliferation disorders such as cancer, immune disorders, inflammation, pain, osteoporosis, atherosclerosis, epilepsy, nausea associated with chemotherapy treatments, fibrosis, gastro-intestinal disorders, neurodegenerative diseases including multiple sclerosis and dyskinesia, Parkinson's disease, Huntington's chorea, Alzheimer's disease but also for preventing or curing diseases associated with motor function such as Tourette's syndrome, and providing neuroprotection.

A subject of the invention is therefore compounds of general formula (I)

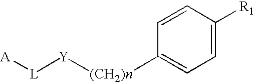

in racemic or enantionieric form or any combinations of these forms and in which

A represents the A₁ or A₂ radical below

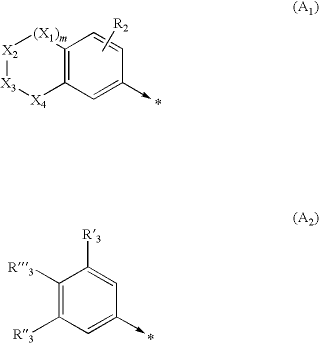

X₁, X₂, X₃ and X₄ represent, independently, an oxygen or sulphur atom, or a radical of formula —NR_{N}— or —C(R₄R₅)— (it being understood that the chain —(X₁)ₘ—X₂-X₃-X₄- does not contain the adjacent heteroatom);

m represents 0 or 1;

R₄ and R₅ represent, independently, the hydrogen atom or a (C₁-C₆)alkyl radical optionally substituted by one or more identical or different halos, or together form the oxo radical;

R_{N} represents the hydrogen atom, a (C₁-C₆)alkyl or (C₁-C₆)alkyl-carbonyl radical;

R₂ represents the hydrogen atom or a (C₁-C₆)alkyl radical;

R′₃, R″₃ and R′″₃ represent, independently, the hydrogen atom, a hydroxy, (C₁-C₆)alkyl or (C₁-C₄)alkoxy radical;

L represents either —C(O)—O— or a radical corresponding to the oxadiazole, oxazole or thiadiazole ring if A represents the (A₁) radical, or a radical corresponding to the oxadiazole, oxazole or thiadiazole ring if A represents the (A₂) radical;

Y represents a covalent bond or the —NH— radical;

n represents 1, 2 or 3;

R₁ represents the —NR′_{N}—C(O)—R′₁; —NR′_{N}—S(O)₂—R′₁; —NR′_{N}—C(Z)-NHR′₁; —C(O)—NH—R′₁, or —N═C(NH₂)R′₁ radical;

R′_{N} represents the hydrogen atom or a (C₁-C₄)alkyl radical;

Z represents the sulphur or oxygen atom;

R′₁ represents a (C₁-C₈)alkyl, (C₂-C₆)alkenyl, (C₁-C₈)hydroxyalkyl, (C₃-C₇)cycloalkyl, spiro-cycloalkyl, (C₃-C₇)heterocycloalkyl, heteroaryl, (C₁-C₈)alkoxy, (C₁-C₈)alkoxy-(C₁-C₈)alkyl radical, or a (CH₂)ₚ—R′₂ radical, all these radicals being optionally substituted by one or more identical or different substituents chosen from: halo, (C₁-C₆)alkyl, (C₁-C₆)haloalkyl;

p represents 1, 2 or 3;

R′₂ represents a (C₃-C₇)cycloalkyl, (C₃-C₇)heterocycloalkyl, aryl or heteroaryl radical, all these radicals being optionally substituted by one or more identical or different substituents chosen from: halo, (C₁-C₆)alkyl, (C₁-C₄)alkoxy and (C₁-C₆)haloalkyl;

it being understood that
i) when L represents —C(O)—O—, then Y represents a covalent bond;
ii) at least one of the R′₃, R″₃ or R′″₃ radicals is different from the hydrogen atom;
iii) when R′″₃ represents a hydroxy or (C₁-C₄)alkoxy radical, then R₁ represents the —NR′_{N}—C(O)—R′₁; —NR′_{N}—S(O)₂—R′₁, or —NR′_{N}—C(Z)-NHR′₁ radical;
   or a pharmaceutically acceptable salt thereof.

In the definitions indicated above, the expression halo represents the fluoro, chloro, bromo or iodo, preferably chloro, fluoro or bromo radical. The expression (C₁-C₈)alkyl (unless otherwise specified), preferably represents an alkyl radical having 1 to 8 carbon atoms, linear or branched, such as the methyl, ethyl, propyl, isopropyl, butyl, isobutyl, sec-butyl and tert-butyl, pentyl or amyl, isopentyl, neopentyl, hexyl or isohexyl, heptyl or octyl radicals. The expression (C₁-C₆)alkyl represents an alkyl radical having 1 to 6 carbon atoms as defined above. The expression (C₁-C₄)alkyl represents an alkyl radical having 1 to 4 carbon atoms as defined above.

By alkenyl, unless otherwise specified, is meant a linear or branched hydrocarbon radical containing 2 to 6 carbon atoms and having at least one unsaturation (double bond), such as for example vinyl, allyl, propenyl, butenyl or pentenyl.

By haloalkyl is meant an alkyl radical at least one (and optionally all) of the hydrogen atoms of which is replaced by a halogen atom (halo) such as for example trifluoromethyl. The term hydroxyalkyl designates the radicals in which the alkyl radical is as defined above and at least one carbon atom of which is substituted by a hydroxy radical such as for example hydroxymethyl, hydroxyethyl, 2-hydroxy-butyl. The term alkyl-carbonyl (or alkyl-C(O)—) designates the radicals in which the alkyl radical is as defined above for example methylcarbonyl, ethylcarbonyl, butylcarbonyl.

The term alkoxy designates the radicals in which the alkyl radical is as defined above such as for example the methoxy, ethoxy, propyloxy or isopropyloxy radicals but also secondary or tertiary linear butoxy, pentyloxy. By (C₁-C₈)alkoxy-(C₁-C₈)alkyl is meant a radical in which the alkoxy and alkyl radicals are as defined above such as for example methoxy-ethyl, methoxy-methyl, ethoxy-ethyl.

The term (C₃-C₇)cycloalkyl designates a saturated monocyclic carbon-containing system comprising 3 to 7 carbon atoms, namely the cyclopropyl, cyclobutyl, cyclopentyl, cyclohexyl or cycloheptyl rings. The expression (C₃-C₇)heterocycloalkyl designates a condensed saturated monocyclic or bicyclic system containing 3 to 7 carbon atoms and at least one heteroatom. This radical can contain several identical or different heteroatoms. Preferably, the heteroatoms are chosen from oxygen, sulphur or nitrogen. As examples of heterocycloalkyl, the following rings can be mentioned: azetidine, pyrrolidine, imidazolidine, pyrrazolidine, isothiazolidine, thiazolidine, isoxazolidine, oxazolidine, piperidine, piperazine, morpholine, azepane (azacycloheptane), tetrahydrofuran (tetrahydrofuryl radical), tetrahydropyran, dioxane, dioxolane or tetrahydrothiophene (tetrahydrothienyl radical).

The term spiro-cycloalkyl designates a spirocyclic saturated hydrocarbon system containing 5 to 10 carbon atoms. As examples of spiro-cycloalkyl, spiro[2.2]pentane, spiro[2.3]hexane, spiroheptane (spiro[2.4]heptane or spiro[3.3]heptane), spirooctane (spiro[2.5]octane or spiro[3.4]octane), spirononane (spiro[2.6]nonane, spiro[3.5]nonane, spiro[4.4]nonane) or spirodecane (spiro[2.7]decane, spiro[3.6]decane, spiro[4.5]decane) can be mentioned.

The expression aryl represents an aromatic radical constituted by a condensed ring or rings, such as for example the phenyl, naphthyl, fluorenyl or anthryl radical. The expression heteroaryl designates an aromatic radical, constituted by a condensed ring or rings, with at least one ring containing one or more identical or different heteroatoms chosen from sulphur, nitrogen or oxygen. As examples of heteroaryl radicals, the following radicals can be mentioned: pyrrolyl, imidazolyl, pyrazolyl, isothiazolyl, thiazolyl, isoxazolyl, oxazolyl, oxadiazolyl, triazolyl, thiadiazolyl, pyridyl, pyrazinyl, pyrimidyl, pyridazinyl, quinolyl, isoquinolyl, quinoxalinyl, indolyl, benzotriazolyl, benzothiazolyl, benzoxadiazoyl, carbazolyl, phenoxazinyl, thieno-pyridinyl (thieno[2,3-b]pyridine, thieno[3,2-b]pyridine, thieno[2,3-c]pyridine, thieno[3,2-c]pyridine, thieno[3,4-b]pyridine, thieno[3,4-c]pyridine), thieno-pyrazinyl (thieno[2,3-b]pyrazine, thieno[3,4-b]pyrazine), thienyl, benzothienyl, furyl, benzofuryl, dihydrobenzofuryl, thioxanthenyl, pyranyl, benzopyranyl, dibenzopyrazinyl, acridinyl.

A more particular subject of the present invention is compounds of formula I as defined above, characterized in that A represents the (A₁) radical.

A more particular subject of the present invention is compounds of formula I as defined above, characterized in that
X₁, X₂, X₃ and X₄ represent, independently, an oxygen atom, or a radical of formula —NR_{N}— or —C(R₄R₅)—;
R_{N} represents the hydrogen atom or a (C₁-C₆)alkyl radical;
R₄ and R₅ represent, independently, the hydrogen atom or a (C₁-C₆) radical alkyl, or together form the oxo radical; and m represents 0 or 1;
   and preferably
X₁ represents an oxygen atom or a radical of formula —NR_{N}— or —C(R₄R₅)—;
X₂ and X₃ represent, independently, the oxygen atom or a radical of formula —C(R₄R₅)—;
X₄ represents an oxygen atom or —C(R₄R₅)—; and m represents 0 or 1.

Very preferably, a subject of the present invention is compounds of formula I as defined above, characterized in that
X₁ represents a radical of formula —NR_{N}— or —C(R₄R₅)—;
X₂ and X₃ represent, independently, a radical of formula —C(R₄R₅)—;

X₄ represents an oxygen atom or —C(R₄R₅)—;

R₄ and R₅ represent, independently, the hydrogen atom or the methyl radical;

R_{N} represents the hydrogen atom or the methyl radical; m represents 0 or 1;

A more particular subject of the present invention is also compounds of formula I as defined above and characterized in that A represents the (A₂) radical.

Preferably, a more particular subject of the present invention is also compounds of formula I as defined above, and characterized in that R′₃, R″₃ and R′″₃ represent, independently, the hydrogen atom or a (C₁-C₆)alkyl radical;

and very preferably
R′₃ and R′₃ represent, independently, the tert-butyl radical, and R′″₃ represents the hydrogen atom.

A more particular subject of the present invention is also compounds of formula I as defined above, and characterized in that n represents 1 or 2.

A more particular subject of the present invention is also compounds of formula I as defined above and characterized in that L represents —C(O)—O—.

A more particular subject of the present invention is also compounds of formula I as defined above, and characterized in that L represents the radical corresponding to the oxadiazole ring.

A more particular subject of the present invention is also compounds of formula I as defined above, and characterized in that L represents the radical corresponding to the oxazole ring.

A more particular subject of the present invention is also compounds of formula I as defined above, and characterized in that L represents the radical corresponding to the thiadiazole ring.

A more particular subject of the present invention is also compounds of formula I as defined above, and characterized in that Y represents a covalent bond and n represents 2.

A more particular subject of the present invention is also compounds of formula I as defined above, and characterized in that
R₁ represents the —NR′_{N}—C(O)—R′¹; —NR′_{N}—S(O)₂—R′₁; —NR′_{N}—C(Z)-NHR′¹; —C(O)—NH—R′₁ or —N═C(NH₂)R′₁, radical;
R′_{N} represents the hydrogen atom;
Z represents the sulphur or oxygen atom;
R′₁ represents a (C₁-C₈)alkyl, (C₂-C₆)alkenyl, (C₃-C₇)Cycloalkyl, spiro-cycloalkyl, (C₃-C₇)heterocycloalkyl, heteroaryl, (C₁-C₈)alkoxy-(C₁-C₈)alkyl radical, or a (CH₂)ₚ—R′₂ radical, all these radicals being optionally substituted by one or more identical or different (C₁-C₆)alkyl substituents;
R′₂ represents a (C₃-C₇)cycloalkyl, (C₃-C₇)heterocycloalkyl, aryl or heteroaryl radical;
   and preferably R₁ represents the —NR′_{N}—C(O)—R′¹; —NR′_{N}—S(O)₂—R′₁ and —NR′_{N}—C(Z)—NHR′₁ radical; and R′_{N} represents the hydrogen atom.

Preferably also
R₁ represents the —NR′_{N}—C(O)—R′₁ or —NR′_{N}—C(Z)-NHR′₁ radical; and R′_{N} represents the hydrogen atom;
Z represents the oxygen atom;
R′₁ represents a (C₁-C₈)alkyl, (C₂-C₆)alkenyl, (C₃-C₇)cycloalkyl radical optionally substituted by one or more identical or different (C₁-C₆)alkyl substituents, spiro-cycloalkyl, or a (CH₂)ₚ—R′₂ radical with p which represents 1;
R′₂ represents a (C₃-C₇)cycloalkyl or heteroaryl radical.

Very preferably, a subject of the present invention is compounds of formula I as defined above and characterized in that
R′₁, represents the —NR′_{N}—C(O)—R′₁ radical; and R′_{N} represents the hydrogen atom;
R′₁ represents a (C₁-C₆)alkyl, (C₂-C₆)alkenyl; or (C₃-C₇)cycloalkyl radical chosen from cyclopropyl, cyclobutyl and cyclopentyl and optionally substituted by one or more identical or different (C₁-C₆)alkyl substituents; spiro[2:3]hexane; or a (CH₂)ₚ—R′₂ radical with p which represents 1;
R′₂ represents a (C₃-C₇)cycloalkyl radical chosen from cyclopropyl, cyclobutyl and cyclopentyl.

Very preferably also, a subject of the present invention is compounds of formula I as defined above and characterized in that
R₁ represents the —NR′_{N}—C(Z)-NHR′₁ radical; R′_{N} represents the hydrogen atom; and Z represents the oxygen atom;
R′₁ represents a (C₁-C₆)alkyl or (C₂-C₆)alkenyl radical.

In the present application, the symbol ->* corresponds to the attachment point of the radical. When the attachment site is not specified on the radical, this means that the attachment is carried out on one of the sites of this radical available for such an attachment.

According to the definitions of the variable A, L, Y and R₁ groups, the compounds according to the invention can be prepared according to the procedures A to N described below:

### Preparation According to Reaction Diagram A

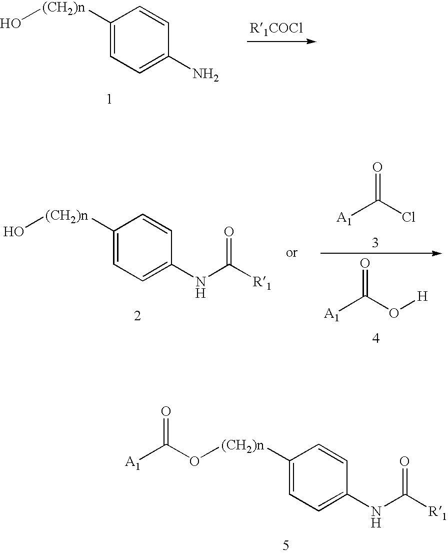

As described in Diagram A, the aniline derivative (1) can be coupled to an acid chloride in the presence of a tertiary base such as triethylamine or diisopropylethylamine at a temperature of approximately 0° C. in order to produce the corresponding amide (2). The alcohol (2) can then be either coupled to an acid chloride (3), in the presence of a tertiary base such as triethylamine or diisopropylethylamine at ambient temperature, or coupled to an acid (4) in the presence of a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI), with or without 1-hydroxybenzotriazole (HOBt), or with Mukaiyama's reagent (2-chloro-1-methyl-pyridinium chloride) in the presence of a tertiary base such as triethylamine or diisopropylethylamine, in an inert organic solvent such as methylene chloride, tetrahydrofuran or dimethylformamide at ambient temperature for 3 to 24 hours or alternatively heated under microwaves at a temperature of 80 to 120° C. (Biotage® equipment), in a sealed tube, for 10 to 30 minutes, in order to produce the corresponding ester (5).

### EXAMPLE A1

### 2-{4-[(cyclobutylcarbonyl)amino]phenyl}ethyl 5,5,8,8-tetramethyl-5,6,7,8-tetrahydronaphthalene-2-carboxylate

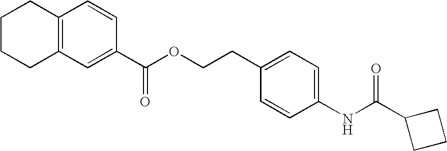

### Stage 1: N-[4-(2-hydroxyethyl)phenyl]cyclobutanecarboxamide

Triethylamine (2.4 mL) and cyclobutane carbonyl chloride (1.7 g) are successively added to a solution cooled down to 0° C. of 2-(4-aminophenyl)ethanol (2 g) in anhydrous dichloromethane (20 mL). After stirring for 2 hours at 0° C., dichloromethane and water are added to the mixture. After decantation and extractions, the combined organic phases are washed with salt water, dried over Na₂SO₄ and concentrated under reduced pressure at 40° C. Purification by flash chromatography on silica gel (eluent: 100% heptane to heptane/ethyl acetate 1:1) produces the expected compound in the form of a white powder (1.9 g; 61% yield).

MS/LC: calculated MM=219.3; m/z=220.2 (MH+)

NMR (¹H, 400 MHz, DMSO-d₆): δ 1.80 (m, 1H), 1.91 (m, 1H), 2.08 (m, 2H), 2.21 (m, 2H), 2.64 (t, 2H), 3.19 (m, 1H), 3.55 (m, 2H), 4.57 (t, 1H), 7.10 (AB, 1H), 7.47 (AB, 1H), 9.59 (s, 1H).

### Stage 2: 2-{4-[(cyclobutylcarbonyl)amino]phenyl}ethyl-5,5,8,8-tetramethyl-5,6,7,8-tetrahydronaphthalene-2-carboxylate

Triethylamine (45 μL, 3 eq), 5,6,7,8-tetrahydronaphthalene-2-carboxylic acid (20 mg, 1.05 eq), Mukaiyama's reagent (2-chloro-1-methyl-pyridinium chloride) supported on polystyrene resin (load: 1.24 mmol/g; 161 mg, 2 eq) then 4-DMAP supported on resin (load: 1.65 mmol/g; 12 mg, 0.2 eq) are successively added to a solution of N-[4-(2-hydroxyethyl)phenyl]cyclobutanecarboxamide (22 mg) in anhydrous dichloromethane (2 mL). The mixture is stirred at ambient temperature for 3 hours then filtered. The filtrate is concentrated under reduced pressure then purified by flash chromatography on silica gel (eluent: 100% heptane to heptane/ethyl acetate 7:3) in order to produce the expected compound in the form of a white powder (20 mg; 55% yield).

MS/LC: calculated MM=377.5; m/z=378.3 (MH+)

NMR (¹H, 400 MHz, DMSO-d₆): δ 1.72 (s, 4H), 1.80 (m, 1H), 1.91 (m, 1H), 2.08 (m, 2H), 2.20 (m, 2H), 2.75 (s, 4H), 2.94 (t, 2H), 3.17 (m, 1H), 4.39 (t, 2H), 7.18 (m, 3H), 7.52 (AB, 2H), 7.60 (m, 2H), 9.62 (s, 1H).

In a fashion analogous to the procedure described for 2-{4-[(cyclobutylcarbonyl)amino]phenyl}ethyl 5,5,8,8-tetramethyl-5,6,7,8-tetrahydronaphthalene-2-carboxylate, the following compounds were prepared:

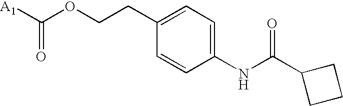

in which A₁ represents one of the radicals below:

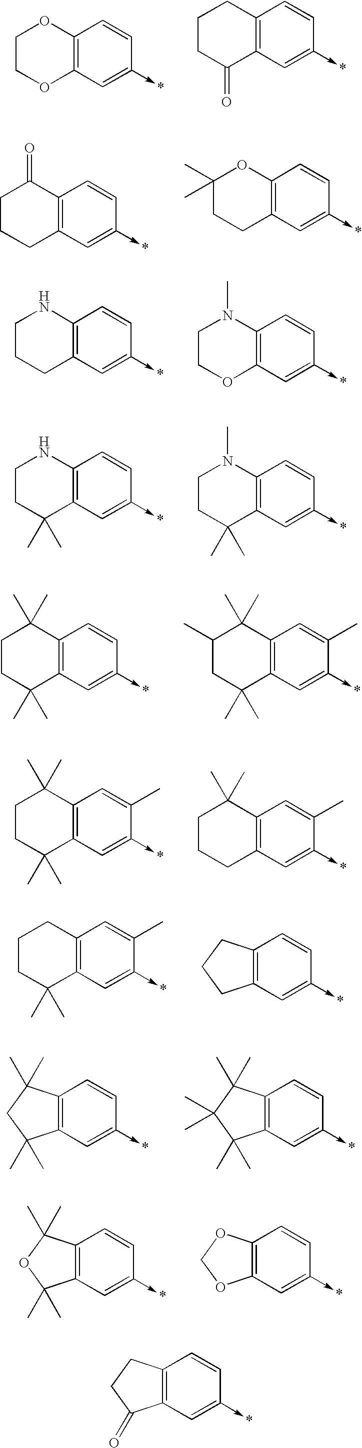

### Preparation According to Reaction Diagram B

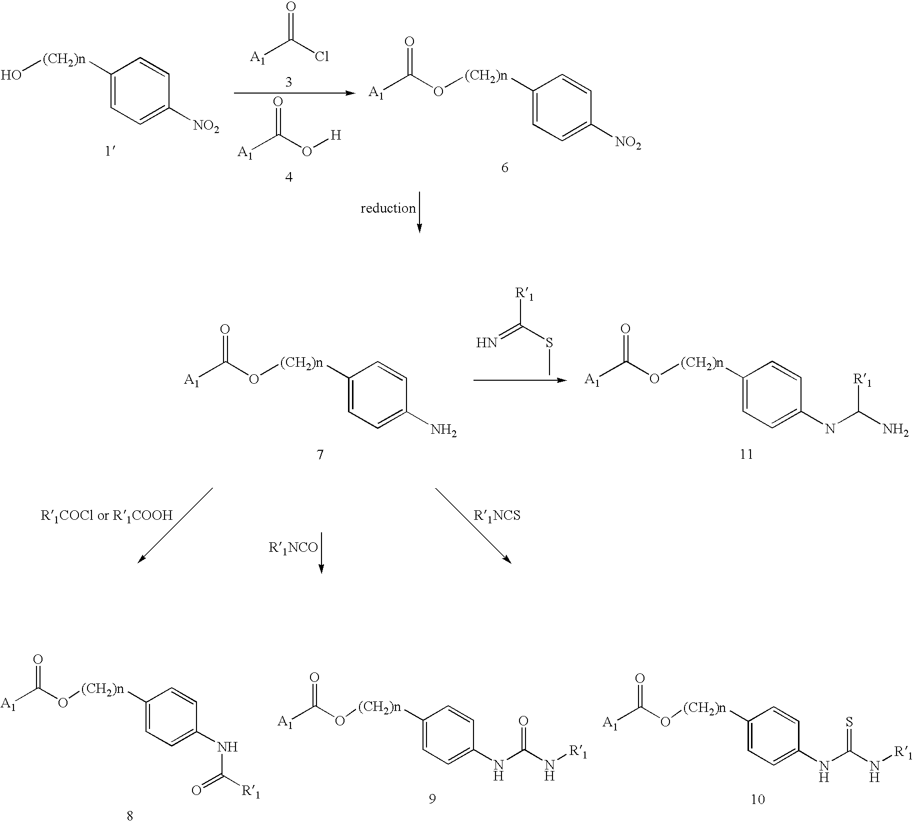

As described in Diagram B, the alcohol (1′) can be either coupled to an acid chloride (3) in the presence of a tertiary base such as triethylamine or disopropylethylamine at ambient temperature, or coupled to an acid (4) in the presence of a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI), with or without 1-hydroxybenzotriazole (HOBt), or with Mukaiyama's reagent (2-chloro-1-methyl-pyridinium chloride) in the presence of a tertiary base such as triethylamine or diisopropylethylamine, in an inert organic solvent such as methylene chloride, tetrahydrofuran or dimethylformamide at ambient temperature for 3 to 24 hours or alternatively heated under microwaves at a temperature of 80 to 120° C. (Biotage® equipment), in a sealed tube, for 10 to 30 minutes, in order to produce the corresponding ester (6). The nitro function of compound (6) is reduced by treatment with dihydrated tin chloride in an inert solvent such as ethyl acetate or dimethylformamide at a temperature of 60-80° C. for 3 to 15 hours or heated under microwaves at a temperature of 80 to 120° C. (Biotage® equipment), in a sealed tube, for 10 to 30 minutes, or alternatively by catalytic hydrogenation in the presence of 100% palladium on carbon in an inert solvent such as methanol, ethanol, ethyl acetate or a mixture of these solvents, at a temperature of 18-25° C., for 2 to 8 hours in order to produce the aniline (7). The aniline (7) can then be either coupled to an acid chloride in the presence of a tertiary base such as triethylamine or diisopropylethylamine at a temperature of approximately 0° C. to 25° C. for 30 min to 3 hours, or coupled to an acid in the presence of a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI), with or without 1-hydroxybenzotriazole (HOBt), or with Mukaiyama's reagent (2-chloro-1-methyl-pyridinium chloride) in the presence of a tertiary base such as triethylamine or diisopropylethylamine, in an inert organic solvent such as methylene chloride, tetrahydrofuran or dimethylformamide at ambient temperature for 3 to 24 hours or alternatively heated under microwaves at a temperature of 80 to 120° C. (Biotage® equipment), in a sealed tube, for 10 to 30 minutes, in order to produce the corresponding amide (8). The aniline (7) can react with an isocyanate or an isothiocyanate in an inert organic solvent such as methylene chloride or tetrahydrofuran at a temperature of 20 to 60° C. in order to produce respectively the urea (9) and the thiourea (10). The aniline (7) can also be treated with a thioimidate in a polar solvent such as methanol or ethanol or DMF at a temperature of 20 to 80° C. for 2 to 24 hours in order to produce the corresponding amidine (11).

### EXAMPLE B1

### 2-{4-[(cyclobutylcarbonyl)amino]phenyl}ethyl 5,5,8,8-tetramethyl-5,6,7,8-tetrahydronaphthalene-2-carboxylate

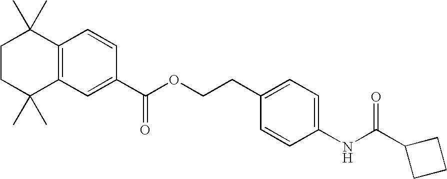

### Stage 1: 2-(4-nitrophenyl)ethyl 5,5,8,8-tetramethyl-5,6,7,8-tetrahydronaphthalene-2-carboxylate

Triethylamine (400 μL) and 5,5,8,8-tetramethyl-5,6,7,8-tetrahydronaphthalene-2-carbonyl chloride (730 mg) are successively added to a solution of 2-(4-nitrophenyl)ethanol (400 mg) in anhydrous dichloromethane (5 ml). After stirring for 2 hours at ambient temperature, water and dichloromethane are added to the mixture. After decantation and extractions, the combined organic phases are washed with salt water, dried over Na₂SO₄ and concentrated under reduced pressure at 40° C. Purification by flash chromatography on silica gel (eluent: 100% heptane to heptane/ethyl acetate 7:3) produces the expected compound in the form of a white powder (515 mg; 57% yield).

NMR (¹H, 400 MHz, DMSO-d₆): δ 1.20 (s, 6H), 1.23 (s, 6H), 1.64 (s, 4H), 3.19 (t, 2H), 4.51 (t, 2H), 7.43 (AB, 1H), 7.59 (m, 3H), 7.75 (s, 1H), 8.17 (AB, 2H).

### Stage 2: 2-(4-aminophenyl)ethyl 5,5,8,8-tetramethyl-5,6,7,8-tetrahydronaphthalene-2-carboxylate

2-(4-nitrophenyl)ethyl 5,5,8,8-tetramethyl-5,6,7,8-tetrahydronaphthalene-2-carboxylate (510 mg) in solution in an ethyl acetate/methanol mixture (1:1; 20 mL) and 10% palladium on carbon (50 mg) are introduced into an autoclave. After stirring for 5 hours under a hydrogen atmosphere (4 bar) at a temperature of approximately 20° C., the catalyst is eliminated by filtration on celite and the filtrate is concentrated under reduced pressure then purified by flash chromatography on silica gel (eluent: 100% heptane to heptane/ethyl acetate 7:3) in order to produce the expected compound in the form of a colourless oil (250 mg; 53% yield).

MS/LC: calculated MM=351.5; m/z=352.3 (MH+)

NMR (¹H, 400 MHz, DMSO-d₆): δ 1.25 (s, 12H), 1.66 (s, 4H), 2.80 (t, 2H), 4.32 (t, 2H), 4.87 (s, 2H), 6.48 (AB, 2H), 6.94 (AB, 2H), 7.46 (AB, 1H), 7.63 (AB, 1H), 7.85 (s, 1H).

### Stage 3: 2-{4-[(cyclobutylcarbonyl)amino]phenyl}ethyl 5,5,8,8-tetramethyl-5,6,7,8-tetrahydronaphthalene-2-carboxylate

Triethylamine (230 μL, 1.2 eq) and cyclobutylcarbonyl chloride (425 mg, 1.2 eq) are successively added to a solution of 2-(4-aminophenyl)ethyl 5,5,8,8-tetramethyl-5,6,7,8-tetrahydronaphthalene-2-carboxylate (300 mg) in THF (8 mL). The mixture is stirred for 2 hours at ambient temperature then concentrated under reduced pressure. Dichloromethane and water are added to the residue. After decantation and extraction, the organic phases are combined, washed twice with salt water, dried over Na₂SO₄ then concentrated under reduced pressure at 40° C. Purification of the residue by flash chromatography on silica gel (eluent: 100% heptane to heptane/ethyl acetate 7:3) produces the expected compound in the form of a white powder (460 mg; 77% yield).

MS/LC: calculated MM=433.6; m/z=434.3 (MH+)

NMR (¹H, 400 MHz, DMSO-d₆): δ 1.23 (s, 12H), 1.64 (s, 4H), 1.80 (m, 1H), 1.92 (m, 1H), 2.07 (m, 2H), 2.20 (m, 2H), 2.94 (t, 2H), 3.19 (m, 1H), 4.40 (t, 2H), 7.21 (AB, 2H), 7.44 (AB, 1H), 7.51 (AB, 2H), 7.61 (AB, 1H), 7.82 (AB, 1H), 9.63 (s, 1H).

### EXAMPLE B2

### 2-(4-{[(propylamino)carbonyl]amino}phenyl)ethyl 5,5,8,8-tetramethyl-5,6,7,8-tetrahydronaphthalene-2-carboxylate

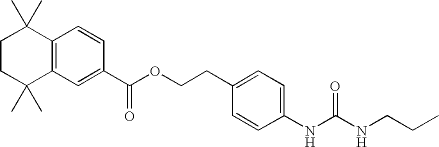

1-isocyanatopropane (36 mg, 1.5 eq) is added to a solution of 2-(4-aminophenyl)ethyl 5,5,8,8-tetramethyl-5,6,7,8-tetrahydronaphthalene-2-carboxylate (100 mg) in THF (1 mL). After stirring for 18 hours at ambient temperature, the mixture is concentrated under reduced pressure then purified by flash chromatography on silica gel (eluent: 100% heptane to heptane/ethyl acetate 6:4) in order to produce the expected compound in the form of a white powder (105 mg; 86% yield).

MS/LC: calculated MM=436.6; m/z=437.3 (MH+)

NMR (¹H, 400 MHz, DMSO-d₆): δ 0.85 (t, 3H), 1.23 (s, 12H), 1.41 (q, 2H), 1.65 (s, 4H), 2.92 (t, 2H), 3.02 (q, 1H), 4.39 (t, 2H), 6.05 (t, 1H), 7.14 (AB, 2H), 7.30 (AB, 2H), 7.45 (AB, 1H), 7.62 (AB, 1H), 7.85 (s, 1H), 8.29 (s, 1H).

### EXAMPLE B3

### 2-(4-{[(ethylamino)carbonothioyl]amino}phenyl)ethyl 5,5,8,8-tetramethyl-5,6,7,8-tetrahydronaphthalene-2-carboxylate

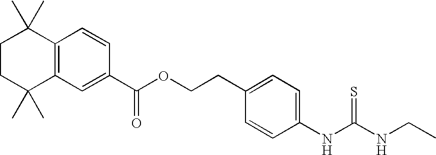

Isothiocyanatoethane (13 mg, 1.5 eq) is added to a solution of 2-(4-aminophenyl)ethyl 5,5,8,8-tetramethyl-5,6,7,8-tetrahydronaphthalene-2-carboxylate (35 mg) in THF (1 mL). After stirring for 18 hours at ambient temperature, the mixture is concentrated under reduced pressure then purified by flash chromatography on silica gel (eluent: 100% heptane to heptane/ethyl acetate 1:1) in order to produce the expected compound in the form of a white powder (36 mg; 82% yield).

MS/LC: calculated MM=438.2; m/z=439.3 (MH+)

NMR (¹H, 400 MHz, DMSO-d₆): δ 1.09 (t, 3H), 1.24 (s, 12H), 1.65 (s, 4H), 2.98 (t, 2H), 3.36 (m, 2H), 4.42 (t, 2H), 7.26 (AB, 2H), 7.31 (AB, 2H), 7.46 (AB, 1H), 7.63 (m, 2H), 7.85 (s, 1H), 9.34 (s, 1H).

In a fashion analogous to the procedure described for 2-(4-{[(propylamino)carbonyl]amino}phenyl)ethyl 5,5,8,8-tetramethyl-5,6,7,8-tetrahydronaphthalene-2-carboxylate, 2-{4-[(cyclobutylcarbonyl)amino]phenyl}ethyl-5,5,8,8-tetramethyl-5,6,7,8-tetrahydronaphthalene-2-carboxylate and 2-(4-{[(ethylamino)carbonothioyl]amino}phenyl)ethyl 5,5,8,8-tetramethyl-5,6,7,8-tetrahydronaphthalene-2-carboxylate, the following compounds were prepared:

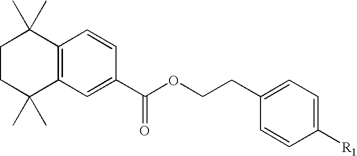

in which R₁ represents one of the radicals below:

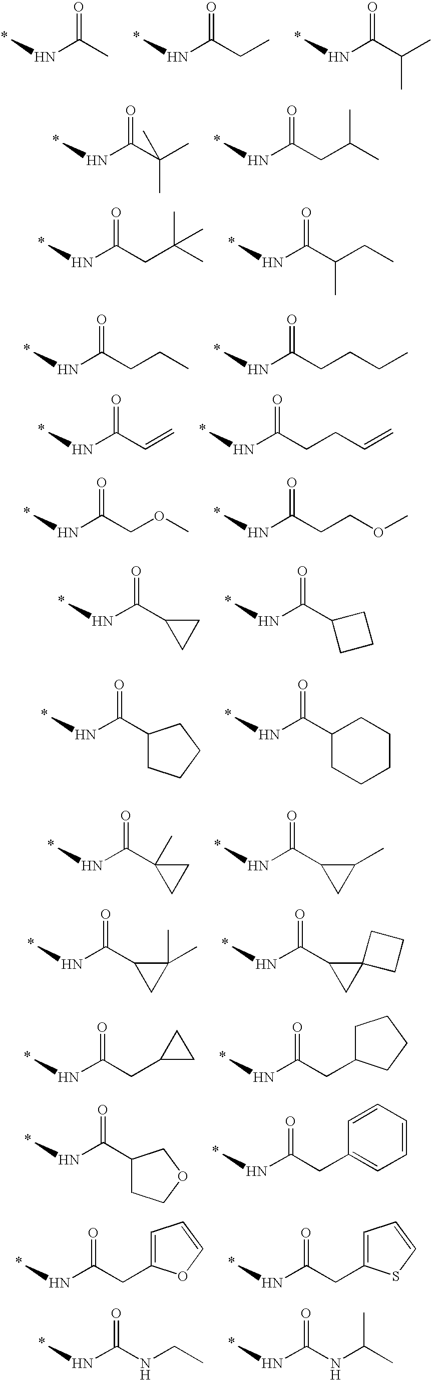

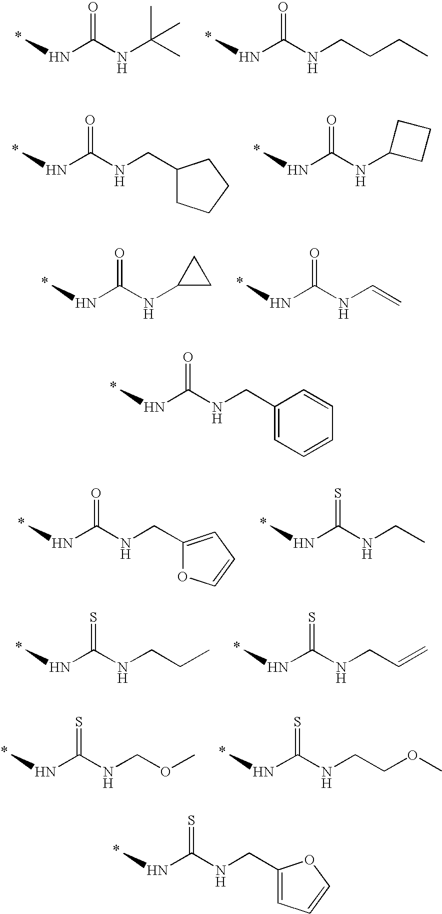

### Preparation According to Reaction Diagram C

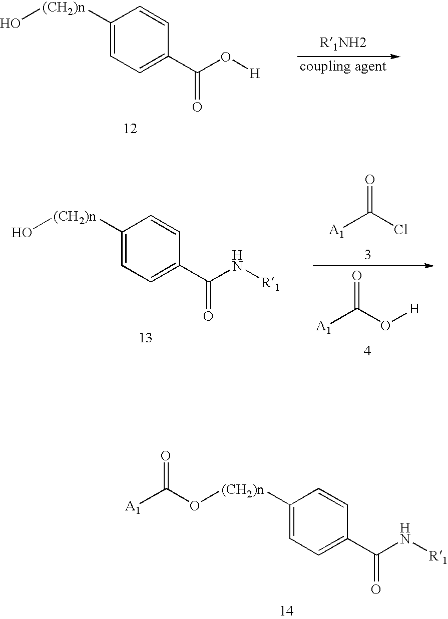

As described in Diagram C, the acid (12) can be coupled to an amine in the presence of a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI), with or without 1-hydroxybenzotriazole (HOBt), or with Mukaiyama's reagent (2-chloro-1-methyl-pyridinium chloride) in the presence of a tertiary base such as triethylamine or diisopropylethylamine, in an inert organic solvent such as methylene chloride, tetrahydrofuran or dimethylformamide at ambient temperature for 3 to 24 hours or alternatively heated under microwaves at a temperature of 80 to 120° C. (Biotage® equipment), in a sealed tube, for 10 to 30 minutes, in order to produce the corresponding amide (13). The alcohol (13) is then either coupled to an acid chloride (3) in the presence of a tertiary base such as triethylamine or disopropylethylamine at ambient temperature, or coupled to an acid (4) in the presence of a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI), with or without 1-hydroxybenzotriazole (HOBt), or with Mukaiyama's reagent (2-chloro-1-methyl-pyridinium chloride) in the presence of a tertiary base such as triethylamine or diisopropylethylamine, in an inert organic solvent such as methylene chloride, tetrahydrofuran or dimethylformamide at ambient temperature for 3 to 24 hours or alternatively heated under microwaves at a temperature of 80 to 120° C. (Biotage® equipment), in a sealed tube, for 10 to 30 minutes, in order to produce the corresponding ester (14).

### EXAMPLE C1

### 4-[(cyclobutylamino)carbonyl]benzyl 5,5,8,8-tetramethyl-5,6,7,8-tetrahydronaphthalene-2-carboxylate

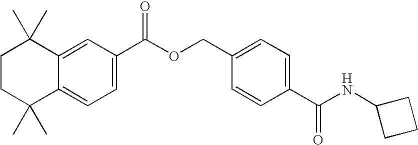

### Stage 1: N-cyclobutyl-4-(hydroxymethyl)benzamide

1-hydroxybenzotriazole (HOBt) (888 mg, 1 eq) and 1-(3-dimethylaminopropyl)-3-ethylcarbodiimide hydrochloride (EDC) (1.26 g, 1 eq) in solution in chloroform (40 ml) then cyclobutylamine (470 mg) are successively added to 4-(hydroxymethyl)benzoic acid (1 g, 1 eq) in solution in anhydrous THF (30 ml). After stirring for 5 hours at a temperature of approximately 20° C., the reaction mixture is concentrated under reduced pressure at 40° C. The residue is taken up in dichloromethane (100 ml) and water (60 ml). After decantation and extractions, the combined organic phases are washed with water, then with salt water, dried over Na₂SO₄ and concentrated under reduced pressure at 40° C. Purification by flash chromatography on silica gel (eluent: heptane/ethyl acetate 40:60 to heptane/ethyl acetate 25:75) produces the expected compound in the form of a white powder (1.3 g; 67% yield).

MS/LC: calculated MM=205.5; m/z=206.2 (MH+)

NMR (¹H, 400 MHz, DMSO-d₆): δ 1.66 (m, 2H), 2.05 (m, 2H), 2.20 (m, 2H), 4.40 (m, 1H), 4.54 (d, 2H), 5.26 (t, 1H), 7.36 (AB, 2H), 2.01 (AB, 2H), 8.52 (d, 1H).

### Stage 2: 4-[(cyclobutylamino)carbonyl]benzyl 5,5,8,8-tetramethyl-5,6,7,8-tetrahydronaphthalene-2-carboxylate

Triethylamine (20 μL) and 5,5,8,8-tetramethyl-5,6,7,8-tetrahydronaphthalene-2-carbonyl chloride (30 mg) are successively added to a solution of N-cyclobutyl-4-(hydroxymethyl)benzamide (20 mg) in anhydrous dichloromethane (2 mL). After stirring for 18 hours at ambient temperature, water and dichloromethane are added to the mixture. After decantation and extractions, the combined organic phases are washed with salt water, dried over Na₂SO₄ and concentrated under reduced pressure at 40° C. Purification by flash chromatography on silica gel (eluent; 100% heptane to heptane/ethyl acetate 70:30) produces the expected compound in the form of a white powder (25 mg; 62% yield).

MS/LC: calculated MM 419.6; m/z=420.3 (MH+)

NMR (¹H, 400 MHz, DMSO-d₆): δ 1.26 (s, 12H), 1.66 (m, 2H), 2.05 (m, 2H), 2.20 (m, 2H), 4.40 (m, 1H), 4.54 (d, 2H), 5.26 (t, 1H), 7.36 (AB, 2H), 2.01 (AB, 2H), 8.52 (d, 1H).

### Preparation According to Reaction Diagram D

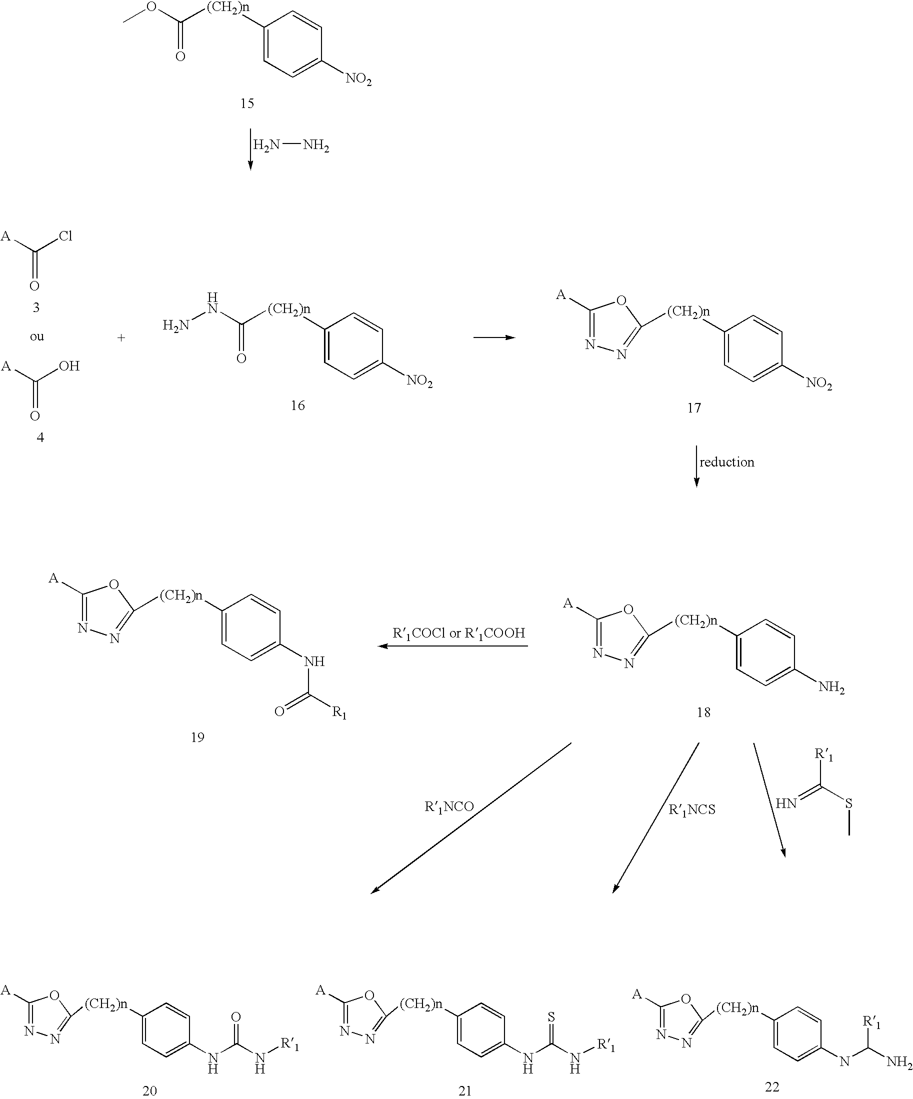

As described in Diagram D, the acid chloride (3) or the acid (4) can be coupled to a hydrazide (16) (either commercial, or prepared by treatment of the corresponding ester (15) with hydrazine in a polar solvent such as methanol or ethanol at ambient temperature for 5 to 24 hours) in the presence of a dehydration agent such as the thionyl chloride, polyphosphoric or sulphuric acid or phosphorus oxychloride used as solvent, or in the presence of Burgess reagent (methyl N(triethylammonium-sulphonyl)carbamate), in an apolar solvent such as tetrahydrofuran, at a temperature of 70 to 120° C. or alternatively heated under microwaves at a temperature of 100 a 150° C. (Biotage® equipment), in a sealed tube, for 10 to 30 minutes, in order to produce the oxadiazole (17). The nitro function of compound (17) is reduced by treatment with dihydrated tin chloride in an inert solvent such as ethyl acetate or dimethylformamide at a temperature of 60-80° C. for 3 to 15 hours or heated under microwaves at a temperature of 100 to 120° C. for 15 to 30 minutes, or alternatively by catalytic hydrogenation in the presence of 10% palladium on carbon in an inert solvent such as ethyl acetate, at a temperature of 18-25° C., for 2 to 8 hours in order to produce the aniline (18). The aniline (18) can then be either coupled to an acid chloride in the presence of a tertiary base such as triethylamine or diisopropylethylamine at a temperature of approximately 0° C. to 25° C. for 30 min to 3 hours, or coupled to an acid in the presence of a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI), with or without 1-hydroxybenzotriazole (HOBt), or with Mukaiyama's reagent (2-chloro-1-methyl-pyridinium chloride) in the presence of a tertiary base such as triethylamine or diisopropylethylamine, in an inert organic solvent such as methylene chloride, tetrahydrofuran or dimethylformamide at ambient temperature for 3 to 24 hours or alternatively heated under microwaves at a temperature of 80 to 120° C. (Biotage® equipment), in a sealed tube, for 10 to 30 minutes, in order to produce the corresponding amide (19). The aniline (18) can react with an isocyanate or an isothiocyanate in an inert organic solvent such as methylene chloride or tetrahydrofuran at ambient temperature in order to produce respectively the urea (20) and the thiourea (21). The aniline (18) can also be treated with a thioimidate in a polar solvent such as methanol or ethanol or DMF at a temperature of 20 to 80° C. for 2 to 24 hours in order to produce the corresponding amidine (22).

### EXAMPLE D1

### N-(4-{2-[5-(5,5,8,8-tetramethyl-5,6,7,8-tetrahydronaphthalen-2-yl)-1,3,4-oxadiazol-2-yl]ethyl}phenyl)cyclobutanecarboxamide

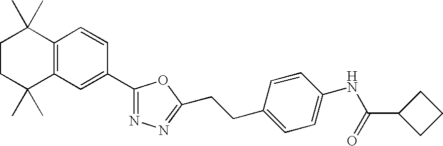

### Stage 1: 2-[2-(4-nitrophenyl)ethyl]-5-(5,5,8,8-tetramethyl-5,6,7,8-tetrahydro naphthalen-2-yl)-1,3,4-oxadiazole

Preparation of 3-(4-nitrophenyl)propanohydrazide: A solution of trimethylsilyldiazomethane (2M in hexane) is added dropwise to a solution cooled down to 0° C. of 3-(4-nitrophenyl)propanoic acid (2.5 g) in methanol (15 mL) until the yellow colouring persists. The mixture is taken to ambient temperature and concentrated under reduced pressure. The solid obtained is purified by flash chromatography on silica gel (eluent: 100% heptane to heptane/ethyl acetate 70:30) in order to produce the expected compound in the form of a white powder (2.5 g, 93% yield).

Monohydrated hydrazine (15 mL) is added to the methyl 3-(4-nitrophenyl)propanoate thus obtained (2.5 g) in solution in ethanol (30 mL). The mixture is stirred for 18 hours at ambient temperature, concentrated under reduced pressure, then water and ethyl acetate are added to it. After decantation and extraction, the organic phases are combined, washed twice with salt water, dried over Na₂SO₄ then concentrated under reduced pressure at 40° C. The solid obtained is washed with diethyl ether in order to produce the expected hydrazide in the form of a white powder (2 g; 80% yield).

MS/LC: calculated MM=209.2; m/z=210.2 (MH+)

NMR (¹H, 400 MHz, DMSO-d₆): δ 2.37 (t, 2H), 2.95 (t, 2H), 4.14 (s, 2H), 7.47 (AB, 2H), 8.13 (AB, 2H), 8.97 (s, 1H).

3-(4-nitrophenyl)propanohydrazide (627 mg) is added to a solution of 5,5,8,8-tetramethyl-5,6,7,8-tetrahydronaphthalene-2-carbonyl chloride (750 mg) in POCl₃ (12 mL) in a “Biotage®” reaction tube. The tube is sealed with a cap, placed in a “Biotage®” micro-wave and heated under magnetic stirring at 150° C. for 1 hour. The mixture is concentrated under reduced pressure then dichloromethane, water and sodium bicarbonate are added until a basic pH is obtained. After decantation and extraction, the organic phases are washed with salt water, dried over Na₂SO₄ then concentrated under reduced pressure at 40° C. Purification of the residue by flash chromatography on silica gel (eluent: 100% heptane to heptane/ethyl acetate 7:3) produces the expected compound in the form of a white powder (430 mg, 35% yield).

MS/LC: calculated MM=405.5; m/z=406.2 (MH+)

NMR (¹H, 400 MHz, DMSO-d₆): δ 1.23; 1.25 (2s, 12H), 1.63 (s, 4H), 3.26 (t, 2H), 3.35 (t, 2H), 7.50 (AB, 1H), 7.60 (AB, 2H), 7.67 (AB, 1H), 7.75 (s, 1H), 8.15 (AB, 2H).

### Stage 2: (4-{2-[5-(5,5,8,8-tetramethyl-5,6,7,8-tetrahydronaphthalen-2-yl)-1,3,4-oxadiazol-2-yl]ethyl}phenyl)amine

2-[2-(4-nitrophenyl)ethyl]-5-(5,5,8,8-tetramethyl-5,6,7,8-tetrahydronaphthalen-2-yl)-1,3,4-oxadiazole (430 mg) in solution in ethyl acetate (20 ml) and 10% palladium on carbon (50 mg) are introduced into an autoclave. After stirring for 5 hours under a hydrogen atmosphere (3 bar) at a temperature of approximately 20° C., the catalyst is eliminated by filtration on celite and the filtrate is concentrated under reduced pressure at 40° C. in order to produce the expected compound in the form of a white powder (372 mg, 93% yield).

MS/LC: calculated MM=375.5; m/z=376.3 (MH+)

NMR (¹H, 400 MHz, DMSO-d₆): δ 1.27 (2s, 12H), 1.67 (s, 4H), 2.91 (t, 2H), 3.13 (t, 2H), 4.85 (s, 2H), 6.46 (AB, 1H), 6.89 (AB, 2H), 7.53 (AB, 1H), 7.68 (AB, 1H), 7.84 (s, 1H).

### Stage 3: N-(4-{2-[5-(5,5,8,8-tetramethyl-5,6,7,8-tetrahydronaphthalen-2-yl)-1,3,4-oxadiazol-2-yl]ethyl}phenyl)cyclobutanecarboxamide

Triethylamine (60 μL, 2 eq) and cyclobutane carbonyl chloride (38 mg, 1.5 eq) are successively added to a solution of (4-{2-[5-(5,5,8,8-tetramethyl-5,6,7,8-tetrahydronaphthalen-2-yl)-1,3,4-oxadiazol-2-yl]ethyl}phenyl)amine (80 mg) in THF (1 mL). The mixture is stirred for 1 hour at ambient temperature then concentrated under reduced pressure. Dichloromethane and water are added to the residue. After decantation and extraction, the organic phases are combined, washed twice with salt water, dried over Na₂SO₄ then concentrated under reduced pressure at 40° C. Purification of the residue by flash chromatography on silica gel (eluent: 100% heptane to heptane/ethyl acetate 6:4) produces the expected compound in the form of a white powder (75 mg, 77% yield).

MS/LC: calculated MM=457.6; m/z=458.3 (MH+)

NMR (¹H, 400 MHz, DMSO-d₆): δ 1.27 (s, 12H), 1.77 (s, 4H), 1.78 (m, 1H), 1.92 (m, 1H), 2.08 (m, 2H), 2.20 (m, 2H), 3.02 (t, 2H), 3.20 (m, 3H), 7.17 (AB, 2H), 7.51 (m, 3H), 7.66 (AB, 1H), 7.78 (s, 1H), 9.62 (s, 1H).

### EXAMPLE D2

### N-allyl-N′-(4-{2-[5-(5,5,8,8-tetramethyl-5,6,7,8-tetrahydronaphthalen-2-yl)-1,3,4-oxadiazol-2-yl]ethyl}phenyl)urea

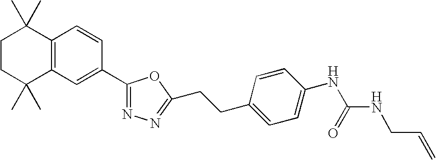

3-isocyanatoprop-1-ene (26 mg, 1.5 eq) is added to a solution of (4-{2-[5-(5,5,8,8-tetramethyl-5,6,7,8-tetrahydronaphthalen-2-yl)-1,3,4-oxadiazol-2-yl]ethyl}phenyl) amine (80 mg) in THF (1 mL). The mixture is stirred for 6 hours at ambient temperature then concentrated under reduced pressure. Purification of the residue by flash chromatography on silica gel (eluent: 100% heptane to heptane/ethyl acetate 1:1) produces the expected compound in the form of a white foam (77 mg; 80% yield).

MS/LC: calculated MM=458.6; m/z=459.3 (MH+)

NMR (¹H, 400 MHz, DMSO-d₆): δ 1.27 (s, 12H), 1.67 (s, 4H), 3.00 (t, 2H), 3.22 (t, 2H), 3.70 (m, 2H), 5.06 (d, 1H), 5.17 (d, 1H), 5.84 (m, 1H), 6.18 (t, 1H), 7.10 (AB, 2H), 7.28 (AB, 2H), 7.52 (AB, 1H), 7.68 (AB, 1H), 7.81 (s, 1H), 8.38 (s, 1H).

### EXAMPLE D3

### N′-(4-{2-[5-(5,5,8,8-tetramethyl-5,6,7,8-tetrahydronaphthalen-2-yl)-1,3,4-oxadiazol-2-yl]ethyl}phenyl)thiophene-2-carboximidamide hydrochloride

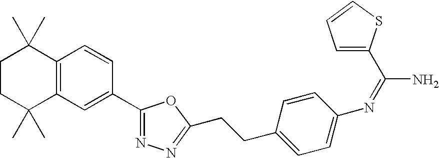

Methyl thiophene-2-carbimidothioate iodide (64 mg, 1.5 eq) is added to a solution of (4-{2-[5-(5,5,8,8-tetramethyl-5,6,7,8-tetrahydronaphthalen-2-yl)-1,3,4-oxadiazol-2-yl]ethyl}phenyl)amine (56 mg) in an isopropanol/THF mixture (1:1; 0.8 mL). The mixture is stirred for 18 hours at ambient temperature then concentrated under reduced pressure. Dichloromethane and a solution of water saturated with hydrogen carbonate are added to the residue. After decantation and extraction, the organic phases are combined, washed with salt water, dried over Na₂SO₄ then concentrated under reduced pressure at 40° C. Purification of the solid by flash chromatography on silica gel (eluent: 100% heptane to heptane/ethyl acetate 4:6) produces the expected compound in the form of a white powder. The corresponding hydrochloride salt is formed by adding a 1N HCl solution in ethyl ether to the solution of the free base in ethyl acetate. The precipitate obtained is filtered and dried in order to produce the expected hydrochloride compound (39 mg, 50% yield).

MS/LC: calculated MM=484.6; m/z=485.3 (MH+)

NMR (¹H, 400 MHz, DMSO-d₆): δ 1.25 (s, 6H), 1.27 (s, 6H), 1.67 (s, 4H), 3.19 (t, 2H), 3.34 (m, 2H), 7.3-8.2 (m, 10H), 8.91 (s, 1H), 9.80 (s, 1H), 11.43 (s, 1H).

### Preparation According to Reaction Diagram E

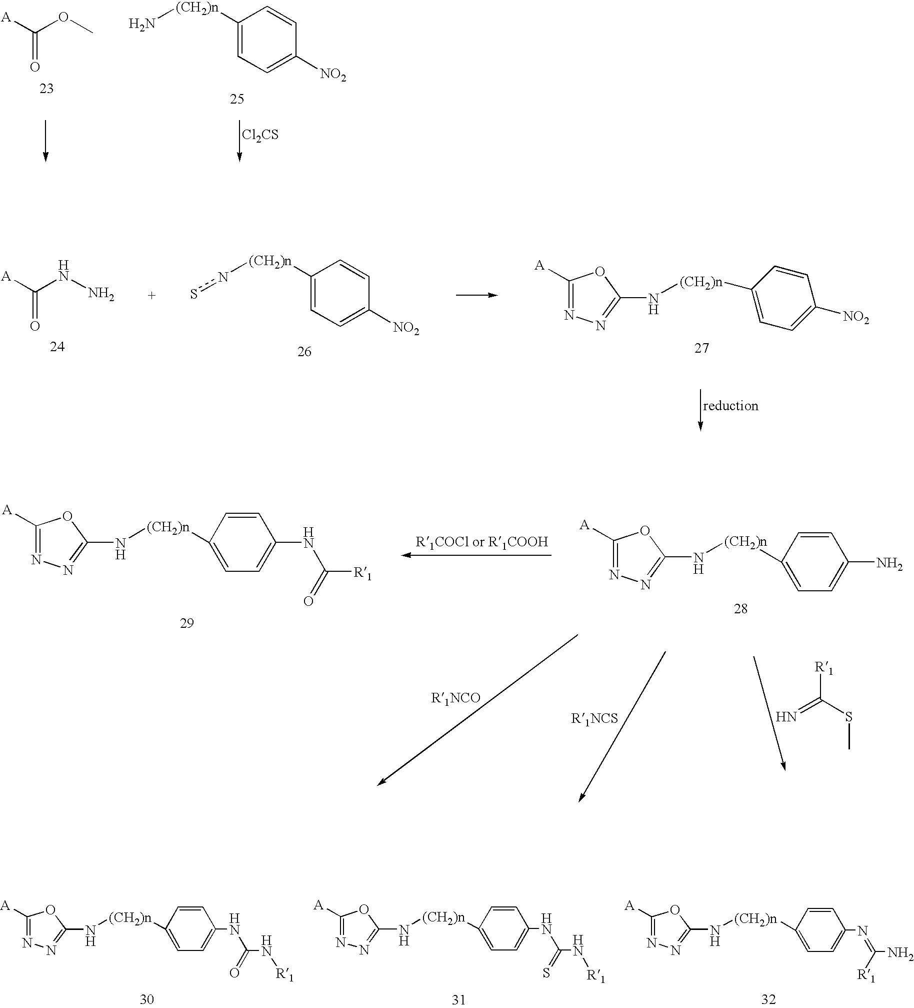

As described in Diagram E, the hydrazide (24) (prepared by treatment of the corresponding ester (23) with hydrazine in a polar solvent such as methanol or ethanol at ambient temperature for 5 to 24 hours) can be coupled to an isothiocyanate (26) (prepared by treatment of the corresponding amine (25) with thiophosgene in an inert solvent such as dichloromethane or tetrahydrofuran at a temperature of 0° C. for 0.2 h to 2 hours) in the presence of a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI) in an aprotic solvent such as tetrahydrofuran, at a temperature of 70 to 120° C. for 2 hours to 24 hours or alternatively mercury oxide, in a polar solvent such as methanol or ethanol, at a temperature of 70 to 80° C. or alternatively heated under microwaves at a temperature of 100 to 120° C. (Biotage® equipment), in a sealed tube, for 10 to 45 minutes, in order to produce the amino-oxadiazole (27). The nitro function of compound (27) is reduced by treatment with dihydrated tin chloride in an inert solvent such as ethyl acetate or dimethylformamide at a temperature of 60-80° C. for 3 to 15 hours or heated under microwaves at a temperature of 100 to 120° C. for 15 to 30 minutes, or alternatively by catalytic hydrogenation in the presence of 10% palladium on carbon in an inert solvent such as ethyl acetate, at a temperature of 18-25° C., for 2 to 8 hours in order to produce the aniline (28). The aniline (28) can then be either coupled to an acid chloride in the presence of a tertiary base such as triethylamine or diisopropylethylamine at a temperature of approximately 0° C. to 25° C. for 30 min to 3 hours, or coupled to an acid in the presence of a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI), with or without 1-hydroxybenzotriazole (HOBt), or with Mukaiyama's reagent (2-chloro-1-methyl-pyridinium chloride) in the presence of a tertiary base such as triethylamine or diisopropylethylamine, in an inert organic solvent such as methylene chloride, tetrahydrofuran or dimethylformamide at ambient temperature for 3 to 24 hours or alternatively heated under microwaves at a temperature of 80 to 120° C. (Biotage® equipment), in a sealed tube, for 10 to 30 minutes, in order to produce the corresponding amide (29). The aniline (28) can react with an isocyanate or an isothiocyanate in an inert organic solvent such as methylene chloride or tetrahydrofuran at ambient temperature in order to produce respectively the urea (30) and the thiourea (31). The aniline (28) can also be treated with a thioimidate in a polar solvent such as methanol or ethanol or DMF at a temperature of 20 to 80° C. for 2 to 24 hours in order to produce the corresponding amidine (32).

### EXAMPLE E1

### N-[4-({[5-(5,5,8,8-tetramethyl-5,6,7,8-tetrahydronaphthalen-2-yl)-1,3,4-oxadiazol-2-yl]amino}methyl)phenyl]cyclobutanecarboxamide

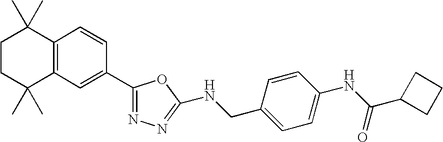

### Preparation of 5,5,8,8-tetramethyl-5,6,7,8-tetrahydronaphthalene-2-carbohydrazide

A solution of trimethylsilyldiazomethane (2M in hexane) is added dropwise to a suspension cooled down to 0° C. of 5,5,8,8-tetramethyl-5,6,7,8-tetrahydronaphthalene-2-carboxylic acid (2 g) in methanol (40 mL) until the yellow colouring persists. The mixture is brought to ambient temperature then concentrated under reduced pressure. The solid obtained is purified by flash chromatography on silica gel (eluent: 100% heptane to heptane/ethyl acetate 85:15) in order to produce the expected compound in the form of a white powder (1.55 g, 73% yield).

Monohydrated hydrazine (15 mL) is added to the methyl 5,5,8,8-tetramethyl-5,6,7,8-tetrahydronaphthalene-2-carboxylate thus obtained (1.5 g) in solution in ethanol (30 mL). The mixture is stirred for 18 hours at ambient temperature, concentrated under reduced pressure, then water and ethyl ether are added to it. After decantation and extraction, the organic phases are combined, washed twice with salt water, dried over Na₂SO₄ then concentrated under reduced pressure at 40° C. Purification of the residue by flash chromatography on silica gel (eluent: 100% heptane to 100% ethyl acetate) produces the expected compound in the form of a white powder (1.3 g; 87% yield).

NMR (¹H, 400 MHz, DMSO-d₆): δ 1.24 (2s, 12H), 1.64 (s, 4H), 4.42 (s, 2H), 7.36 (AB, 1H), 7.55 (AB, 1H), 7.77 (s, 1H), 9.67 (s, 1H).

### Stage 1: N-(4-nitrobenzyl)-5-(5,5,8,8-tetramethyl-5,6,7,8-tetrahydronaphthalen-2-yl)-1,3,4-oxadiazol-2-amine

1-(isothiocyanatomethyl)-4-nitrobenzene (434 mg, 1.1 eq) and mercury oxide (700 mg, 2 eq) are successively added to a solution of 5,5,8,8-tetramethyl-5,6,7,8-tetrahydronaphthalene-2-carbohydrazide (500 mg) in ethanol (10 mL) in a “Biotage®” reaction tube. The tube is sealed with a cap, placed in a “Biotage®” micro-wave and heated under magnetic stirring at 110° C. for 45 minutes. The mixture is then filtered on celite and the filtrate is concentrated under reduced pressure. Purification of the residue by flash chromatography on silica gel (eluent: 100% heptane to heptane/ethyl acetate 1:1) produces the expected compound in the form of a white powder (540 mg; 66% yield).

MS/LC: calculated MM=406.5; m/z=407.2 (MH+)

NMR (¹H, 400 MHz, DMSO-d₆): 1.25 (s, 12H), 1.66 (s, 4H), 4.58 (d, 2H), 7.47 (AB, 1H), 7.53 (AB, 1H), 7.66 (m, 3H), 8.21 (AB, 2H), 8.46 (t, 1H).

### Stage 2: N-(4-aminobenzyl)-5-(5,5,8,8-tetramethyl-5,6,7,8-tetrahydronaphthalen-2-yl)-1,3,4-oxadiazol-2-amine

N-(4-nitrobenzyl)-5-(5,5,8,8-tetramethyl-5,6,7,8-tetrahydronaphthalen-2-yl)-1,3,4-oxadiazol-2-amine (535 mg) in solution in ethyl acetate (20 ml) and 10% palladium on carbon (55 mg) are introduced into an autoclave. After stirring for 4 hours under a hydrogen atmosphere (3 bar) at a temperature of approximately 20° C., the catalyst is eliminated by filtration on celite and the filtrate is concentrated under reduced pressure at 40° C. Purification by flash chromatography on silica gel (eluent: 100% heptane to heptane/ethyl acetate 45:55) produces the expected compound in the form of a white powder (350 mg; 71% yield).

MS/LC: calculated MM=376.5; m/z=377.3 (MH+)

NMR (¹H, 400 MHz, DMSO-d₆): δ 1.26 (2s, 12H), 1.66 (s, 4H), 4.22 (d, 2H), 4.98 (s, 2H), 6.51 (AB, 1H), 7.03 (AB, 2H), 7.46 (AB, 1H), 7.52 (AB, 1H), 7.69 (s, 1H), 8.03 (t, 1H).

### Stage 3: N-[4-({[5-(5,5,8,8-tetramethyl-5,6,7,8-tetrahydronaphthalen-2-yl)-1,3,4-oxadiazol-2-yl]amino}methyl)phenyl]cyclobutanecarboxamide

Triethylamine (42 μL, 2 eq) and cyclobutane carbonyl chloride (20 mg, 1.5 eq) are successively added to a solution of N-(4-aminobenzyl)-5-(5,5,8,8-tetramethyl-5,6,7,8-tetrahydronaphthalen-2-yl)-1,3,4-oxadiazol-2-amine (57 mg) in THF (0.8 mL). The mixture is stirred for 1 hour at ambient temperature then concentrated under reduced pressure. Dichloromethane and water are added to the residue. After decantation and extraction, the organic phases are combined, washed twice with salt water, dried over Na₂SO₄ then concentrated under reduced pressure at 40° C. The solid obtained is washed with diethyl ether in order to produce the expected compound in the form of a white powder (55 mg; 80% yield).

MS/LC: calculated MM=458.6; m/z=459.3 (MH+)

NMR (1H, 400 MHz, DMSO-d₆): δ 1.26 (s, 12H), 1.66 (s, 4H), 1.78 (m, 1H), 1.91 (m, 1H), 2.10 (m, 2H), 2.20 (m, 2H), 3.07 (m, 1H), 4.36 (d, 2H), 7.28 (AB, 2H), 7.54 (m, 3H), 7.68 (s, 1H), 8.20 (t, 1H), 9.69 (s, 1H).

### EXAMPLE E2

### N-propyl-N′-[4-({[5-(5,5,8,8-tetramethyl-5,6,7,8-tetrahydronaphthalen-2-yl)-1,3,4-oxadiazol-2-yl]amino}methyl)phenyl]urea

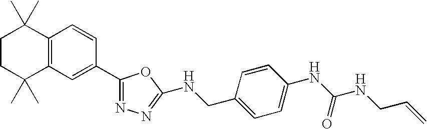

3-isocyanatoprop-1-ene (26 mg, 1.5 eq) is added to a solution of N-(4-aminobenzyl)-5-(5,5,8,8-tetramethyl-5,6,7,8-tetrahydronaphthalen-2-yl)-1,3,4-oxadiazol-2-amine (57 mg) in THF (0.8 mL). The mixture is stirred for 6 hours at ambient temperature then concentrated under reduced pressure. The solid obtained is washed with a dichloromethane/diethyl ether mixture in order to produce the expected compound in the form of a white powder (51 mg; 75% yield).

MS/LC: calculated MM=459.6; m/z=459.3 (MH+)

NMR (1H, 400 MHz, DMSO-d₆): δ 1.25 (s, 6H), 1.26 (s, 6H), 1.66 (s, 4H), 3.71 (t, 2H), 4.33 (d, 2H), 5.06 (d, 1H), 5.14 (d, 1H), 5.81 (m, 1H), 6.20 (t, 1H), 7.23 (AB, 2H), 7.34 (AB, 2H), 7.46 (AB, 1H), 7.53 (AB, 1H), 7.70 (s, 1H), 8.17 (t, 1H), 8.46 (s, 1H).

### Preparation According to Reaction Diagram F

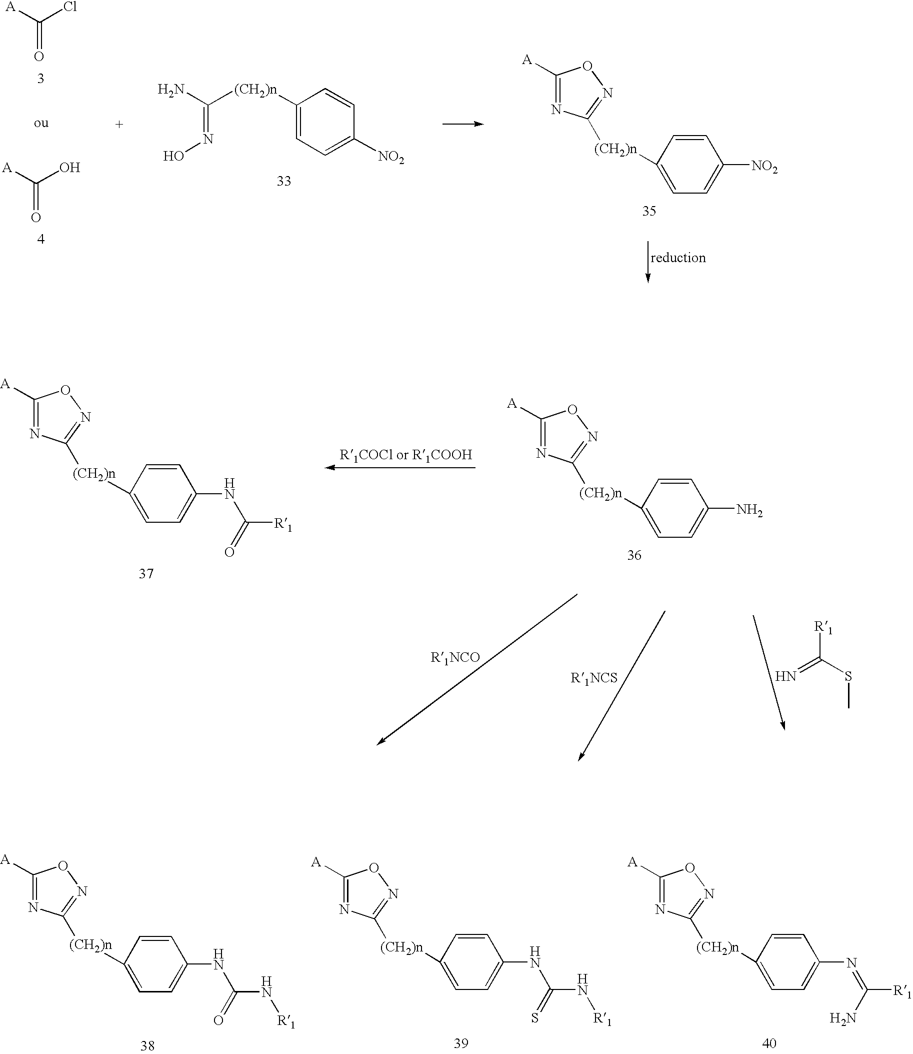

As described in Diagram F, the acid chloride (3) can be coupled to amidoxime (33) (commercial or prepared from the corresponding nitrile derivative by treatment with hydroxylamine in the presence of an inorganic base such as potassium carbonate in a polar solvent such as ethanol, at a temperature of 60 to 80° C. for 1 to 24 hours) in the presence of a tertiary base such as triethylamine or diisopropylethylamine, or the acid (4) can be coupled to amidoxime (33) in the presence of a coupling agent such as diisopropylcarbodiimide (DIC), dicyclohexylcarbodiimide (DCC), 1-(3-dimethylaminopropyl)-3-ethylcarbodiimide hydrochloride (EDC), carbonyldiimidazole (CDI), 1-hydroxybenzotriazole (HOBt), O-benzotriazol-1-yl-N,N,N′,N′-tetramethyluronium terafluoroborate (TBTU) or O-benzotriazol-1-yl-N,N,N′N′-tetramethyluronium hexafluorophosphate (HBTU), in an aprotic solvent such as tetrahydrofuran or acetonitrile, at a temperature of 60 to 90° C. for 8 to 72 hours. Alternatively, the mixture can be heated under microwaves at a temperature of 100 to 150° C. (Biotage® equipment), in a sealed tube, for 30 minutes to 2 hours, in order to produce the corresponding amino-oxadiazole (35). The nitro function of compound (35) is reduced by treatment with dihydrated tin chloride in an inert solvent such as ethyl acetate or dimethylformamide at a temperature of 60-80° C. for 3 to 15 hours or heated under microwaves at a temperature of 100 to 120° C. for 15 to 30 minutes, or alternatively by catalytic hydrogenation in the presence of 10% palladium on carbon in an inert solvent such as ethyl acetate, at a temperature of 18-25° C., for 2 to 8 hours in order to produce the aniline (36). The aniline (36) can then be either coupled to an acid chloride in the presence of a tertiary base such as triethylamine or diisopropylethylamine at a temperature of approximately 0° C. to 25° C. for 30 min to 3 hours, or coupled to an acid in the presence of a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI), with or without 1-hydroxybenzotriazole (HOBt), or with Mukaiyama's reagent (2-chloro-1-methyl-pyridinium chloride) in the presence of a tertiary base such as triethylamine or diisopropylethylamine, in an inert organic solvent such as methylene chloride, tetrahydrofuran or dimethylformamide at ambient temperature for 3 to 24 hours or alternatively heated under microwaves at a temperature of 80 to 120° C. (Biotage® equipment), in a scaled tube, for 10 to 30 minutes, in order to produce the corresponding amide (37). The aniline (36) can react with an isocyanate or an isothiocyanate in an inert organic solvent such as methylene chloride or tetrahydrofuran at ambient temperature in order to produce respectively the urea (38) and the thiourea (39). The aniline (36) can also be treated with a thioimidate in a polar solvent such as methanol or ethanol or DMF at a temperature of 20 to 80° C. for 2 to 24 hours in order to produce the corresponding amidine (40).

### EXAMPLE F1

### N-(4-{[5-(3,5-di-tert-butylphenyl)-1,2,4-oxadiazol-3-yl]methyl}phenyl)cyclobutanecarboxamide

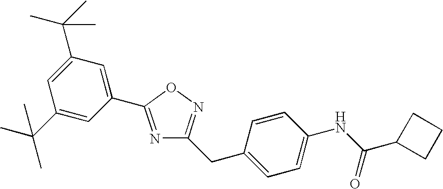

### Stage 1: 1-N′-[(3,5-di-tert-butylbenzoyl)oxy]-2-(4-nitrophenyl)ethanimidamide

1-N′-hydroxy-2-(4-nitrophenyl)ethanimidamide (972 mg, 1 eq), O-benzotriazol-1-yl-N,N,N′N′-tetramethyluronium hexafluorophosphate (HBTU) (1.7 g, 1 eq) and diisopropylethylamine (2.2 mL, 3 eq) are successively added to a solution of 3,5-di-tert-butyl benzoic acid (1.85 g) in acetonitrile (25 mL). The mixture is stirred for 3 hours at ambient temperature then the precipitate formed is washed with diethyl ether in order to produce the expected compound in the form of a white powder (1.3 g, 70% yield).

MS/LC: calculated MM=411.5; m/z=412.3 (MH+)

NMR (¹H, 400 MHz, DMSO-d₆): 1.31 (s, 18H), 3.62 (s, 2H), 6.66 (s, 2H), 7.66 (m, 3H), 7.81 (s, 2H), 8.21 (AB, 2H).

### Stage 2: 5-(3,5-di-tert-butylphenyl)-3-(4-nitrobenzyl)-1,2,4-oxadiazole

A mixture of 1-N′-[(3,5-di-tert-butylbenzoyl)oxy]-2-(4-nitrophenyl)ethanimidamide (1.3 g) in acetonitrile (15 mL) is placed in a “Biotage®” reaction tube. The tube is sealed with a cap, placed in a “Biotage®” micro-wave and heated under magnetic stirring at 140° C. for 2 hours. The mixture is then concentrated under reduced pressure then purified by flash chromatography on silica gel (eluent: 100% heptane to heptane/ethyl acetate 85:15) in order to produce the expected compound in the form of an oil (760 mg; 61% yield).

MS/LC: calculated MM=393.5; m/z=394.2 (MH+)

NMR (¹H, 400 MHz, DMSO-d₆): 1.32 (s, 18H), 4.38 (s, 2H), 7.63 (AB, 2H), 7.73 (s, 1H), 7.86 (s, 2H), 8.20 (AB, 2H).

### Stage 3: 4-{[5-(3,5-di-tert-butylphenyl)-1,2,4-oxadiazol-3-yl]methyl}phenyl)amine

A mixture of 5-(3,5-di-tert-butylphenyl)-3-(4-nitrobenzyl)-1,2,4-oxadiazole (700 mg) and dihydrated tin chloride (2 g, 5 eq) in ethyl acetate (10 mL) is placed in a “Biotage®” reaction tube. The tube is sealed with a cap, placed in a “Biotage®” micro-wave and heated under magnetic stirring at 100° C. for 20 minutes then ethyl acetate and water saturated with hydrogen carbonate are added. After decantation and extraction, the organic phases are combined, washed with salt water, dried over Na₂SO₄ then concentrated under reduced pressure at 40° C. The residue obtained is purified by flash chromatography on silica gel (eluent: 100% heptane to heptane/ethyl acetate 75:25) in order to produce the expected compound in the form of a white solid (540 mg; 83% yield).

MS/LC: calculated MM=363.5; m/z=364.3 (MH+)

NMR (¹H, 400 MHz, DMSO-d₆): 1.32 (s, 18H), 3.93 (s, 2H), 4.98 (s, 2H), 6.50 (AB, 2H), 6.96 (AB, 2H), 7.70 (s, 1H), 7.86 (s, 1H).

### Stage 4: N-(4-{[5-(3,5-di-tert-butylphenyl)-1,2,4-oxadiazol-3-yl]methyl}phenyl)cyclobutanecarboxamide

Triethylamine (42 μL, 2 eq) and cyclobutane carbonyl chloride (27 mg, 1.5 eq) are successively added to a solution of 4-{[5-(3,5-di-tert-butylphenyl)-1,2,4-oxadiazol-3-yl]methyl}phenyl)amine (55 mg) in THF (0.8 mL). The mixture is stirred for 3 hours at ambient temperature then concentrated under reduced pressure. Dichloromethane and water are added to the residue. After decantation and extraction, the organic phases are combined, washed twice with salt water, dried over Na₂SO₄ then concentrated under reduced pressure at 40° C. The solid obtained is purified by flash chromatography on silica gel (eluent: 100% heptane to heptane/ethyl acetate 6:4) in order to produce the expected compound in the form of a white solid (56 mg, 85% yield).

MS/LC: calculated MM=445.6; m/z=446.3 (MH+)

NMR (¹H, 400 MHz, DMSO-d₆): 1.32 (s, 18H), 1.80 (m, 1H), 1.92 (m, 1H), 2.09 (m, 2H), 2.20 (m, 2H), 3.19 (m, 1H), 4.10 (s, 2H), 7.23 (AB, 2H), 7.55 (AB, 2H), 7.72 (s, 1H), 7.85 (s, 2H), 9.68 (s, 1H).

### EXAMPLE F2

### N-(4-{[5-(3,5-di-tert-butylphenyl)-1,2,4-oxadiazol-3-yl]methyl}phenyl)-N′-propylurea

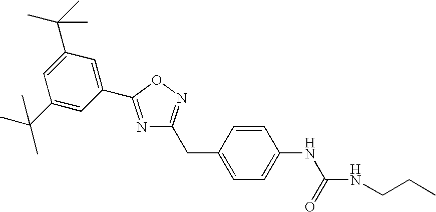

3-isocyanatopropane (20 mg, 1.5 eq) is added to a solution of 4-{[5-(3,5-di-tert-butylphenyl)-1,2,4-oxadiazol-3-yl]methyl}phenyl)amine (55 mg) in THF (0.8 mL). The mixture is stirred for 18 hours at ambient temperature then concentrated under reduced pressure. The solid obtained is washed with a dichloromethane/diethyl ether mixture in order to produce the expected compound in the form of a white powder (45 mg; 68% yield).

MS/LC: calculated MM=448.6; m/z=449.4 (MH+)

NMR (¹H, 400 MHz, DMSO-d₆): 0.85 (t, 3H), 1.33 (s, 18H), 1.42 (q, 2H), 4.06 (s, 2H), 6.08 (t, 1H), 7.16 (AB, 2H), 7.33 (AB, 2H), 7.72 (s, 1H), 7.85 (s, 2H), 8.35 (s, 1H).

### Preparation According to Reaction Diagram G

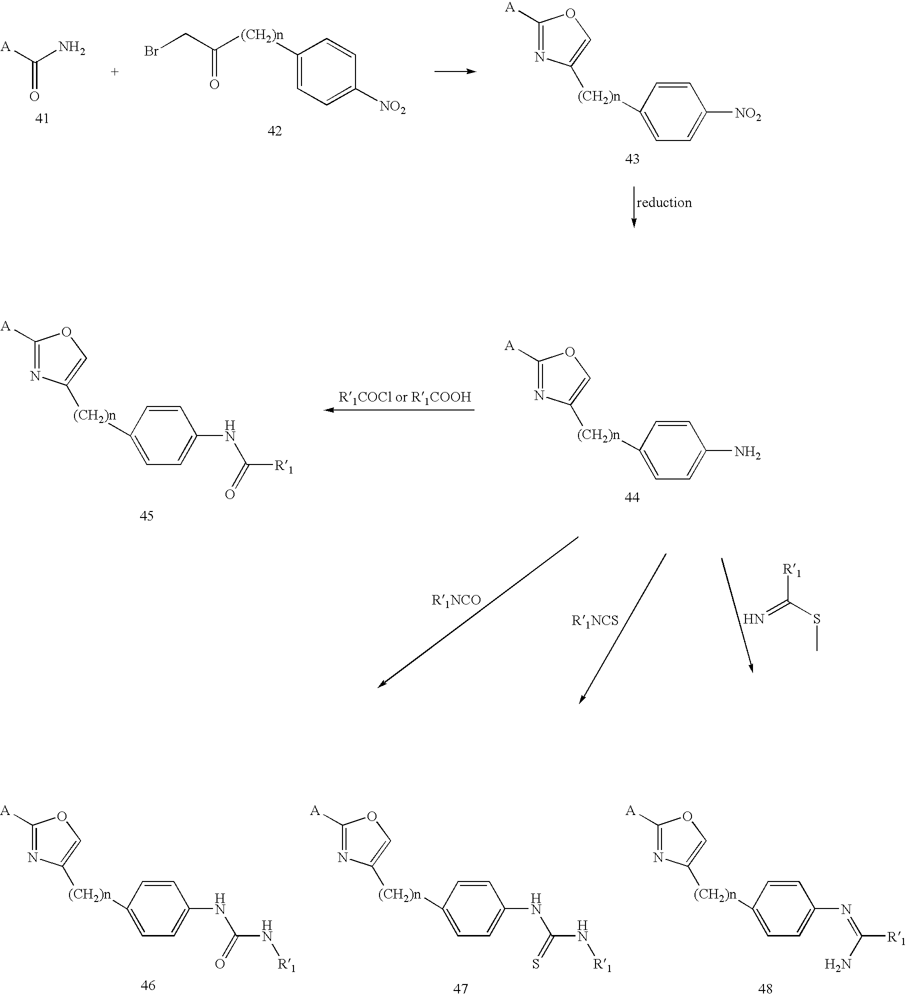

As described in Diagram G, the amide (41) can be coupled to an α-bromoketone (42) in an aprotic solvent such as THF or DMF at a temperature of 80 to 120° C. for 3 to 24 hours or alternatively heated under microwaves at a temperature of 120° C. to 150° C. (Biotage® equipment), in a sealed tube, for 10 to 45 minutes, in order to produce the oxazole (43). The nitro function of compound (43) is reduced by treatment with dihydrated tin chloride in an inert solvent such as ethyl acetate or dimethylformamide at a temperature of 60-80° C. for 3 to 15 hours or heated under microwaves at a temperature of 100 to 120° C. for 15 to 30 minutes, or alternatively by catalytic hydrogenation in the presence of 10% palladium on carbon in an inert solvent such as ethyl acetate, at a temperature of 18-25° C., for 2 to 8 hours in order to produce the aniline (44). The aniline (44) can then be either coupled to an acid chloride in the presence of a tertiary base such as triethylamine or diisopropylethylamine at a temperature of approximately 0° C. to 25° C. for 30 min to 3 hours, or coupled to an acid in the presence of a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI), with or without 1-hydroxybenzotriazole (HOBt), or with Mukaiyama's reagent (2-chloro-1-methyl-pyridinium chloride) in the presence of a tertiary base such as triethylamine or diisopropylethylamine, in an inert organic solvent such as methylene chloride, tetrahydrofuran or dimethylformamide at ambient temperature for 3 to 24 hours or alternatively heated under microwaves at a temperature of 80 to 120° C. (Biotage® equipment), in a scaled tube, for 10 to 30 minutes, in order to produce the corresponding amide (45). The aniline (44) can react with an isocyanate or an isothiocyanate in an inert organic solvent such as methylene chloride or tetrahydrofuran at ambient temperature in order to produce respectively the urea (46) and the thiourea (47). The aniline (44) can also be treated with a thioimidate in a polar solvent such as methanol or ethanol or DMF at a temperature of 20 to 80° C. for 2 to 24 hours in order to produce the corresponding amidine (48).

### Preparation According to Reaction Diagram H

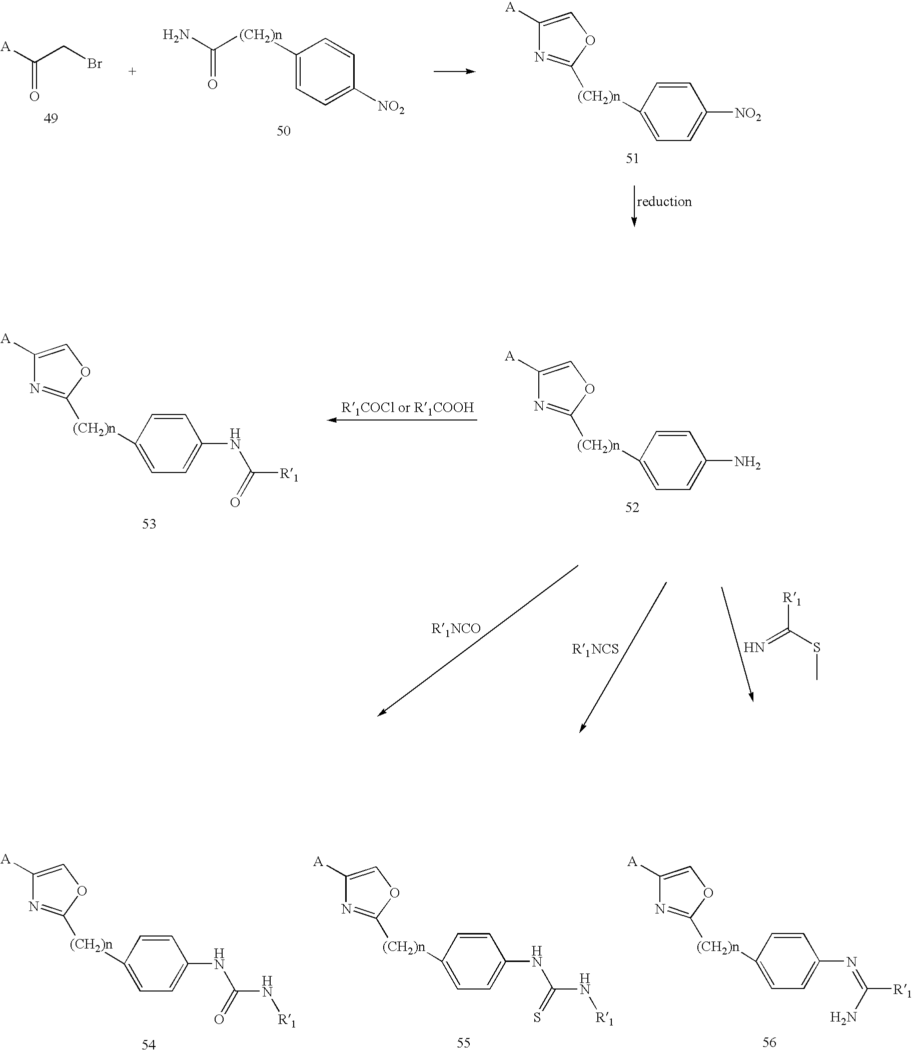

The procedures for Diagram H are analogous to those described for Diagram G.

### EXAMPLE H1

### N-(4-{2-[4-(5,5,8,8-tetramethyl-5,6,7,8-tetrahydronaphthalen-2-yl)-1,3-oxazol-2-yl]ethyl}phenyl)cyclobutanecarboxamide

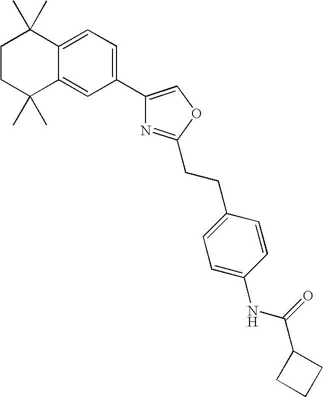

### Stage 1: 2-[2-(4-nitrophenyl)ethyl]-4-(5,5,8,8-tetramethyl-5,6,7,8-tetrahydronaphthalen-2-yl)-1,3-oxazole

3-(4-nitrophenyl)propanamide (470 mg, 1.5 eq) is added to a solution of 2-bromo-1-(5,5,8,8-tetramethyl-5,6,7,8-tetrahydronaphthalen-2-yl)ethanone (500 mg) in DMF in a “Biotage®” reaction tube. The tube is sealed with a cap, placed in a “Biotage®” micro-wave and heated under magnetic stirring at 150° C. for 30 minutes. The reaction medium is concentrated under reduced pressure. The residue obtained is purified by flash chromatography on silica gel (eluent: 100% heptane to heptane/ethyl acetate 9:1) in order to produce the expected compound (260 mg; 40% yield).

MS/LC: calculated MM=404.2; m/z=405.2 (MH+)

NMR (¹H, 400 MHz, DMSO-d₆): δ 1.23-1.25 (s, 12H), 1.64 (s, 4H), 3.18 (m, 4H), 7.33 (AB, 1H), 7.44 (AB, 1H), 7.57 (AB, 2H), 7.64 (s, 1H), 8.14 (AB, 2H), 8.43 (s, 1H).

### Stage 2: (4-{2-[4-(5,5,8,8-tetramethyl-5,6,7,8-tetrahydronaphthalen-2-yl)-1,3-oxazol-2-yl]ethyl}phenyl)amine

A mixture of 2-[2-(4-nitrophenyl)ethyl]-4-(5,5,8,8-tetramethyl-5,6,7,8-tetrahydronaphthalen-2-yl)-1,3-oxazole (250 mg) and dihydrated tin chloride (700 mg, 5 eq) in ethyl acetate (5 mL) is placed in a “Biotage®” reaction tube. The tube is sealed with a cap, placed in a “Biotage®” micro-wave and heated under magnetic stirring at 120° C. for 30 minutes then ethyl acetate and water saturated with hydrogen carbonate are added. After decantation and extraction, the organic phases are combined, washed with salt water, dried over Na₂SO₄ then concentrated under reduced pressure at 40° C. The residue obtained is purified by flash chromatography on silica gel (eluent: 100% heptane to heptane/ethyl acetate 85:15) in order to produce the expected compound (185 mg; 80% yield).

MS/LC: calculated MM=374.2; m/z=375.3 (MH+)

NMR (1H, 400 MHz, DMSO-d₆): δ 1.23-1.26 (2s, 12H), 1.64 (s, 4H), 2.84 (t, 2H), 2.97 (t, 2H), 4.86 (s, 2H), 6.45 (AB, 2H), 6.87 (AB, 2H), 7.33 (AB, 1H), 7.45 (AB, 1H), 7.65 (s, 1H), 8.43 (s, 1H).

### Stage 3: N-(4-{2-[4-(5,5,8,8-tetramethyl-5,6,7,8-tetrahydronaphthalen-2-yl)-1,3-oxazol-2-yl]ethyl}phenyl)cyclobutanecarboxamide

Triethylamine (32 μL, 2 eq) and cyclobutane carbonyl chloride (20 mg, 1.5 eq) are successively added to a solution of (4-{2-[4-(5,5,8,8-tetramethyl-5,6,7,8-tetrahydronaphthalen-2-yl)-1,3-oxazol-2-yl]ethyl}phenyl)amine (43 mg) in THF (0.5 mL). The mixture is stirred for 3 hours at ambient temperature then concentrated under reduced pressure. Dichloromethane and water are added to the residue. After decantation and extraction, the organic phases are combined, washed twice with salt water, dried over Na₂SO₄ then concentrated under reduced pressure at 40° C. The solid obtained is washed with a dichloromethane/diethyl ether mixture in order to produce the expected compound in the form of a white powder (39 mg; 75% yield).

MS/LC: calculated MM=456.3; m/z=457.4 (MH+)

NMR (¹H, 400 MHz, DMSO-d₆): δ 1.24-1.26 (2s, 12H), 1.64 (s, 4H), 1.79 (m, 1H), 1.90 (m, 1H), 2.07 (m, 2H), 2.21 (m, 2H), 2.98 (m, 2H), 3.05 (m, 2H), 3.20 (m, 1H), 7.15 (AB, 2H), 7.33 (AB, 1H), 7.44 (AB, 1H), 7.46 (AB, 2H), 7.65 (s, 1H), 8.41 (s, 1H), 9.60 (s, 1H).

### Preparation According to Reaction Diagram J

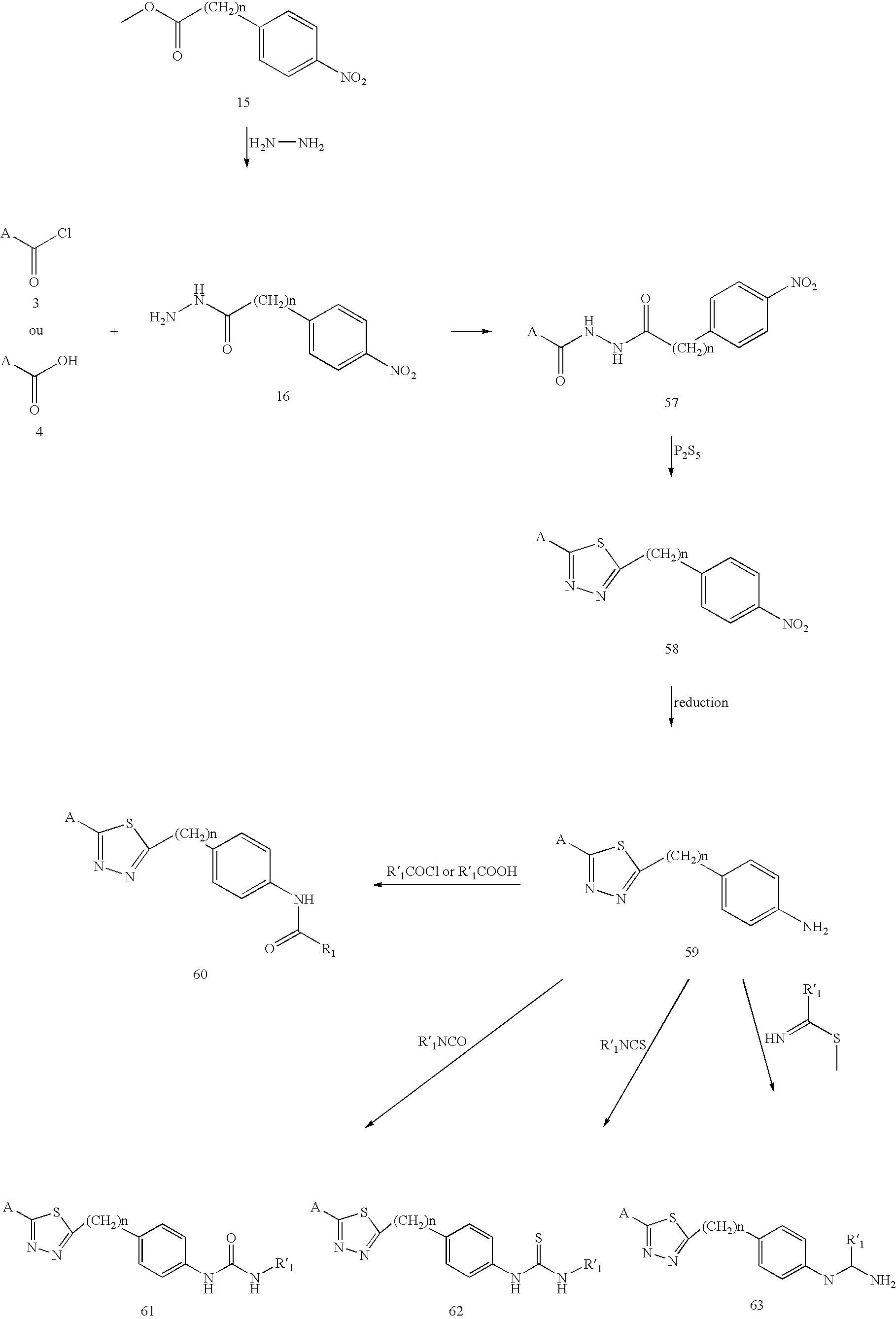

As described in Diagram J, the acid chloride (3) or the acid (4) can be coupled to a hydrazide (15) (either commercial, or prepared by treatment of the corresponding ester (15) with the hydrazine in a polar solvent such as methanol or ethanol at ambient temperature for 5 to 24 hours) in order to produce N,N-diacyl-hydrazine (57). The cyclization in thiadiazole (58) is carried out by treatment with phosphorus (V) sulphide in an inert solvent, such as tetrahydrofuran or acetonitrile, at a temperature of 18 to 80° C., for 2 to 15 hours. The nitro function of the compound (58) is reduced by treatment with dihydrated tin chloride in an inert solvent such as ethyl acetate or dimethylformamide at a temperature of 60-80° C. for 3 to 15 hours or heated under microwaves at a temperature of 100 to 120° C. for 15 to 30 minutes, or alternatively by catalytic hydrogenation in the presence of 10% palladium on carbon in an inert solvent such as ethyl acetate, at a temperature of 18-25° C., for 2 to 8 hours in order to produce the aniline (59). The aniline (59) can then be either coupled to an acid chloride in the presence of a tertiary base such as triethylamine or diisopropylethylamine at a temperature of approximately 0° C. to 25° C. for 30 min to 3 hours, or coupled to an acid in the presence of a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI), with or without 1-hydroxybenzotriazole (HOBt), or with Mukaiyama's reagent (2-chloro-1-methyl-pyridinium chloride) in the presence of a tertiary base such as triethylamine or diisopropylethylamine, in an inert organic solvent such as methylene chloride, tetrahydrofuran or dimethylformamide at ambient temperature for 3 to 24 hours or alternatively heated under microwaves at a temperature of 80 to 120° C. (Biotage® equipment), in a sealed tube, for 10 to 30 minutes, in order to produce the corresponding amide (60). The aniline (59) can react with an isocyanate or an isothiocyanate in an inert organic solvent such as methylene chloride or tetrahydrofuran at ambient temperature in order to produce respectively the urea (61) and the thiourea (62). The aniline (59) can also be treated with a thioimidate in a polar solvent such as methanol or ethanol or DMF at a temperature of 20 to 80° C. for 2 to 24 hours in order to produce the corresponding amidine (63).

### EXAMPLE J1

### N-(4-{2-[5-(3,5-di-tert-butylphenyl)-1,3,4-thiadiazol-2-yl]ethyl}phenyl)-N′-propylurea

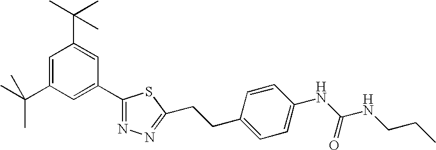

### Stage 1: 3,5-di-tert-butyl-N′-[3-(4-nitrophenyl)propanoyl]benzohydrazide

HBTU (1.2 g, 1 eq), diisopropylethylamine (1.6 mL, 1 eq) and 3-(4-nitrophenyl)propanohydrazide (680 mg, 1 eq) are successively added to 3,5-di-tert-butylbenzoic acid (750 mg) in THF (15 mL). The mixture is stirred for 6 hours at ambient temperature then concentrated under reduced pressure. The solid is dissolved in dichloromethane (300 mL) then water is added (100 mL). After decantation and extraction, the organic phases are combined, dried over Na₂SO₄ then concentrated under reduced pressure at 40° C. The solid obtained is washed with a dichloromethane/diethyl ether mixture in order to produce the expected compound in the form of a white powder (1.1 g; 75% yield).

MS/LC: calculated MM=425.2; m/z=426.3 (MH+)

NMR (1H, 400 MHz, DMSO-d₆): δ 1.30 (s, 18H), 2.59 (t, 2H), 3.01 (t, 2H), 7.49 (AB, 2H), 7.50 (s, 1H), 7.69 (s, 2H), 8.16 (AB, 2H), 9.92 (s, 1H), 10.3 (s, 1H).

### Stage 2: 2-(3,5-di-tert-butylphenyl)-5-[2-(4-nitrophenyl)ethyl]-1,3,4-thiadiazole

Phosphorus (V) sulphide (640 mg, 2 eq) is added to a solution of 3,5-di-tert-butyl-N′-[3-(4-nitrophenyl)propanoyl]benzohydrazide (615 mg) in anhydrous THF. The mixture is stirred for 7 hours at ambient temperature then ethyl acetate and a saturated solution of hydrogen carbonate are added. After decantation and extraction, the organic phases are combined, dried over Na₂SO₄ then concentrated under reduced pressure at 40° C. The solid obtained is purified by flash chromatography on silica gel (eluent: 100% heptane to heptane/ethyl acetate 7:3) in order to produce the expected compound (430 mg; 70% yield).

MS/LC: calculated MM=423.6; m/z=424.2 (MH+)

NMR (¹H, 400 MHz, DMSO-d₆): δ 1.32 (s, 18H), 3.27 (t, 2H), 3.52 (t, 2H), 7.59 (m, 3H), 7.67 (s, 2H), 8.15 (AB, 2H).

### Stage 3: (4-{2-[5-(3,5-di-tert-butylphenyl)-1,3,4-thiadiazol-2-yl]ethyl}phenyl)amine

A mixture of 2-(3,5-di-tert-butylphenyl)-5-[2-(4-nitrophenyl)ethyl]-1,3,4-thiadiazole (425 mg) and dihydrated tin chloride (1.1 g, 5 eq) in ethyl acetate (5 mL) is placed in a “Biotage®” reaction tube. The tube is sealed with a cap, placed in a “Biotage®” micro-wave and heated under magnetic stirring at 120° C. for 30 minutes then ethyl acetate and water saturated with hydrogen carbonate are added. After decantation and extraction, the organic phases are combined, washed with salt water, dried over Na₂SO₄ then concentrated under reduced pressure at 40° C. The residue obtained is purified by flash chromatography on silica gel (eluent: 100% heptane to heptane/ethyl acetate 7:3) in order to produce the expected compound (330 mg, 84% yield).

MS/LC: calculated MM=393.2; m/z=394.2 (MH+)

NMR (¹H, 400 MHz, DMSO-d₆): δ 1.33 (s, 18H), 3.90 (t, 2H), 3.33 (t, 2H), 4.86 (s, 2H), 6.47 (AB, 2H), 6.91 (AB, 2H), 7.58 (s, 1H), 7.67 (s, 2H).

### Stage 4: N-(4-{2-[5-(3,5-di-tert-butylphenyl)-1,3,4-thiadiazol-2-yl]ethyl}phenyl)-N′-propylurea

3-isocyanatopropane (26 mg, 1.5 eq) is added to a solution of (4-{2-[5-(3,5-di-tert-butylphenyl)-1,3,4-thiadiazol-2-yl]ethyl}phenyl)amine (78 mg) in THF (1 mL). The mixture is stirred for 18 hours at ambient temperature then concentrated under reduced pressure. The solid obtained is washed with diethyl ether in order to produce the expected compound in the form of a white powder (51 mg; 75% yield).

MS/LC: calculated MM=478.3; m/z ˜479.3 (MH+)

NMR (¹H, 400 MHz, DMSO-d₆): δ 0.85 (t, 3H), 1.33 (s, 18H), 1.40 (t, 2H), 3.00 (m, 4H), 3.37 (t, 2H), 6.06 (s, 1H), 7.11 (AB, 2H), 7.28 (AB, 2H), 7.58 (s, 1H), 7.67 (s, 2H), 8.29 (s, 1H).

### Preparation According to Reaction Diagram K

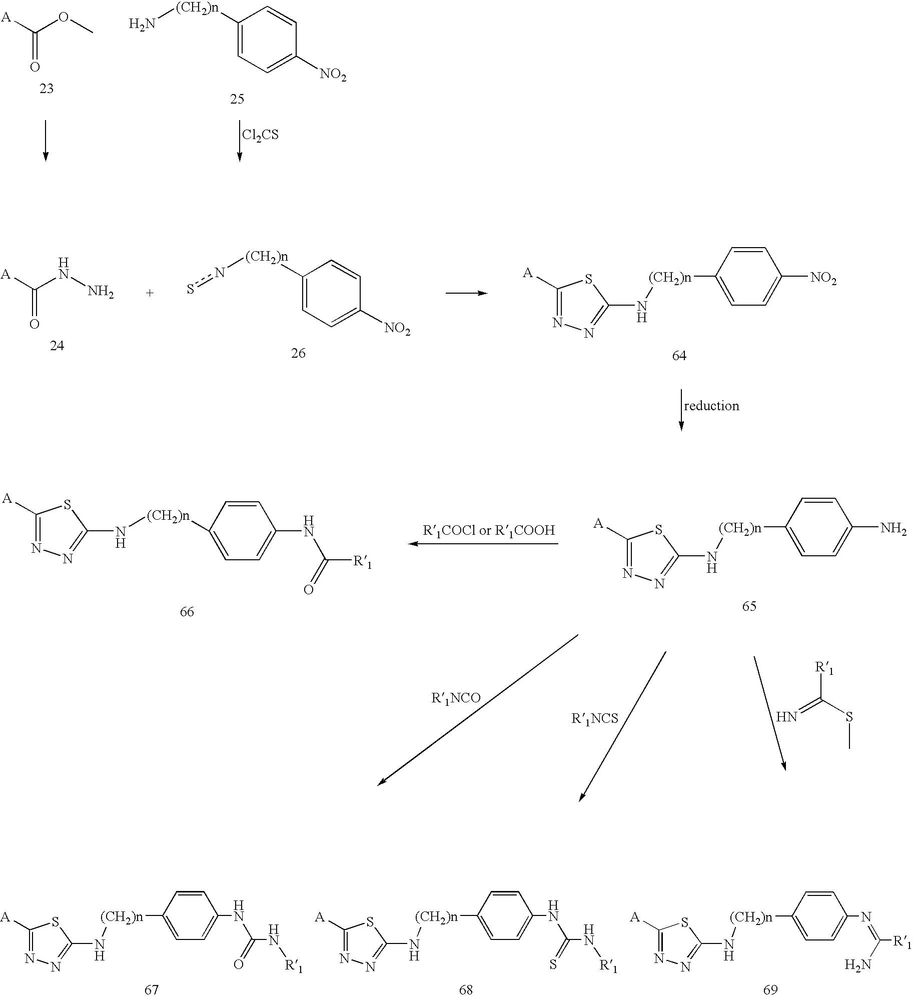

As described in Diagram K, the hydrazide (24) (prepared by treatment of the corresponding ester (23) with hydrazine in a polar solvent such as methanol or ethanol at ambient temperature for 5 to 24 hours) can be coupled to an isothiocyanate (26) (prepared by treatment of the corresponding amine (25) with thiophosgene in an inert solvent such as dichloromethane or tetrahydrofuran at a temperature of 0° C. for 0.2 h to 2 hours) in a polar solvent such as methanol, ethanol, dimethylacetamide, in the presence or absence of an organic acid such as acetic acid or an inorganic acid such as phosphoric acid at a temperature of 70 to 120° C. for 2 to 24 hours or alternatively heated under microwaves at a temperature of 100 to 120° C. (Biotage® equipment), in a sealed tube, for 10 to 45 minutes, in order to produce the amino-thiadiazole (64). The nitro function of compound (64) is reduced by treatment with dihydrated tin chloride in an inert solvent such as ethyl acetate or dimethylformamide at a temperature of 60-80° C. for 3 to 15 hours or heated under microwaves at a temperature of 100 to 120° C. for 15 to 30 minutes, or alternatively by catalytic hydrogenation in the presence of 10% palladium on carbon in an inert solvent such as ethyl acetate, at a temperature of 18-s 25° C., for 2 to 8 hours in order to produce the aniline (65). The aniline (65) can then be either coupled to an acid chloride in the presence of a tertiary base such as triethylamine or diisopropylethylamine at a temperature of approximately 0° C. to 25° C. for 30 min to 3 hours, or coupled to an acid in the presence of a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI), with or without 1-hydroxybenzotriazole (HOBt), or with Mukaiyama's reagent (2-chloro-1-methyl-pyridinium chloride) in the presence of a tertiary base such as triethylamine or diisopropylethylamine, in an inert organic solvent such as methylene chloride, tetrahydrofuran or dimethylformamide at ambient temperature for 3 to 24 hours or alternatively heated under microwaves at a temperature of 80 to 120° C. (Biotage® equipment), in a sealed tube, for 10 to 30 minutes, in order to produce the corresponding amide (66). The aniline (65) can react with an isocyanate or an isothiocyanate in an inert organic solvent such as methylene chloride or tetrahydrofuran at ambient temperature in order to produce respectively the urea (67) and the thiourea (68). The aniline (65) can also be treated with a thioimidate in a polar solvent such as methanol or ethanol or DMF at a temperature of 20 to 80° C. for 2 to 24 hours in order to produce the corresponding amidine (69).

### Preparation According to Reaction Diagram L

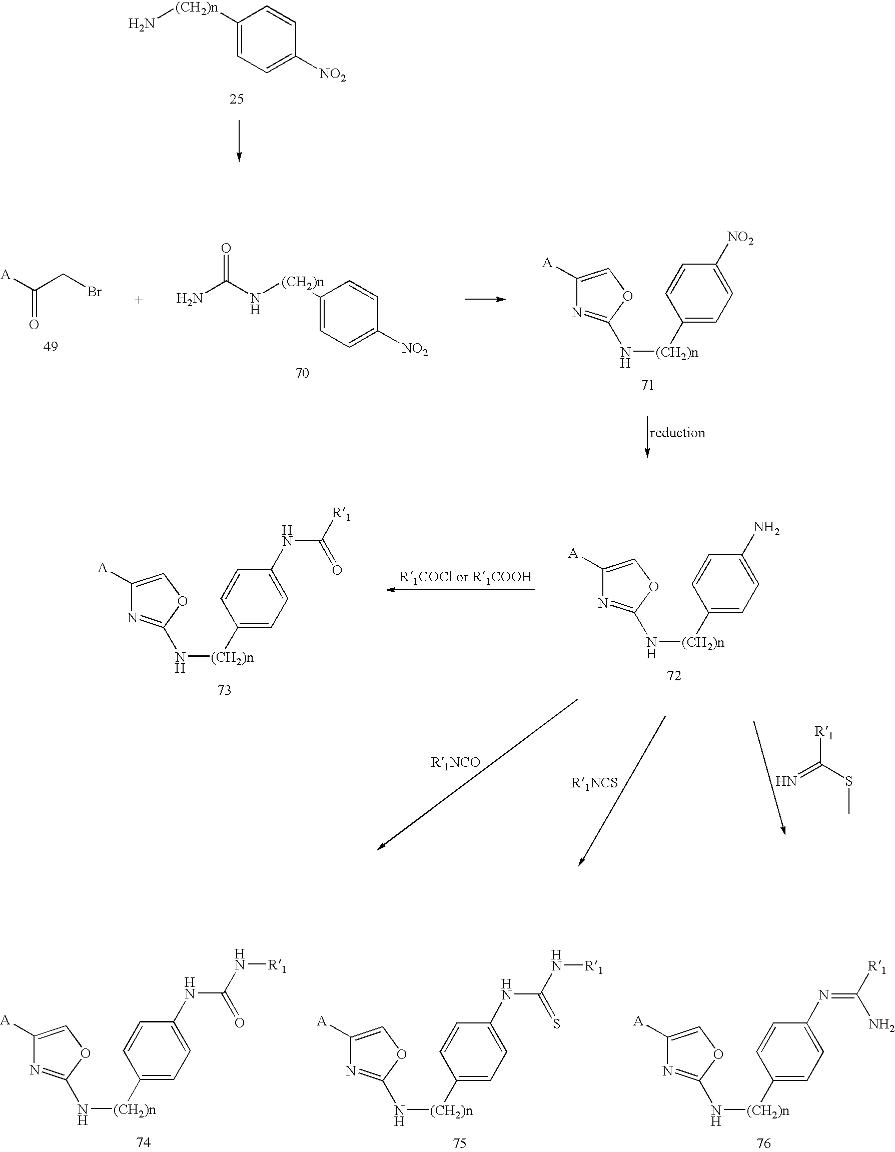

As described in Diagram L, the urea (70) (prepared from the corresponding amine (25), by treatment with urea in aqueous hydrochloric acid at a temperature of 20-110° C. for 2 to 24 hours) can be coupled to an α-bromoketone (49) in a protic solvent such as water in the presence of an acid such as hydrochloric acid at a temperature of 20 to 110° C. for 3 to 24 hours or alternatively heated under microwaves at a temperature of 120° C. to 150° C. (Biotage® equipment), in a sealed tube, for 10 to 45 minutes, in order to produce the amino-oxazole (71). The nitro function of compound (71) is reduced by treatment with dihydrated tin chloride in an inert solvent such as ethyl acetate or dimethylformamide at a temperature of 60-80° C. for 3 to 15 hours or heated under microwaves at a temperature of 100 to 120° C. for 15 to 30 minutes, or alternatively by catalytic hydrogenation in the presence of 10% palladium on carbon in an inert solvent such as ethyl acetate, at a temperature of 18-25° C., for 2 to 8 hours in order to produce the aniline (72). The aniline (72) can then be either coupled to an acid chloride in the presence of a tertiary base such as triethylamine or diisopropylethylamine at a temperature of approximately 0° C. to 25° C. for 30 min to 3 hours, or coupled to an acid in the presence of a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI), with or without 1-hydroxybenzotriazole (HOBt), or with Mukaiyama's reagent (2-chloro-1-methyl-pyridinium chloride) in the presence of a tertiary base such as triethylamine or diisopropylethylamine, in an inert organic solvent such as methylene chloride, tetrahydrofuran or dimethylformamide at ambient temperature for 3 to 24 hours or alternatively heated under microwaves at a temperature of 80 to 120° C. (Biotage® equipment), in a sealed tube, for 10 to 30 minutes, in order to produce the corresponding amide (73). The aniline (72) can react with an isocyanate or an isothiocyanate in an inert organic solvent such as methylene chloride or tetrahydrofuran at ambient temperature in order to produce respectively the urea (74) and the thiourea (75). The aniline (72) can also be treated with a thioimidate in a polar solvent such as methanol or ethanol or DMF at a temperature of 20 to 80° C. for 2 to 24 hours in order to produce the corresponding amidine (76).

### Preparation According to Reaction Diagram M

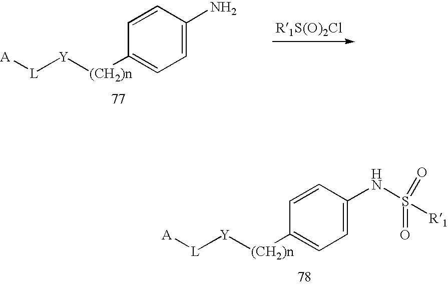

As described in Diagram M, the aniline (77) can react with a sulphonyl chloride, in an aprotic organic solvent such as dichloromethane or THF, in the presence of a tertiary base such as triethylamine, at a temperature of 0 to 60° C., for 1 to 24 hours, in order to produce the corresponding sulphonamide (78).

### EXAMPLE M1

### 2-{4-[(methylsulphonyl)amino]phenyl}ethyl 5,5,8,8-tetramethyl-5,6,7,8-tetrahydronaphthalene-2-carboxylate

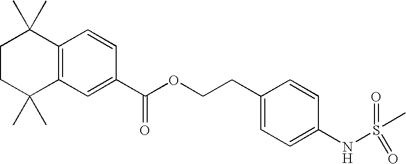

Triethylamine (12 mg) and methyl sulphonyl chloride (14 mg; 1.2 eq) are successively added to a solution of 2-(4-aminophenyl)ethyl 5,5,8,8-tetramethyl-5,6,7,8-tetrahydronaphthalene-2-carboxylate (prepared according to example B1) (35 mg) in anhydrous dichloromethane (1 mL). The mixture is stirred for 4 hours at ambient temperature then washed with water and salt water. The organic phase is dried over Na₂SO₄ then concentrated under reduced pressure at 40° C. The residue obtained is purified by flash chromatography on silica gel (eluent: 100% heptane to heptane/ethyl acetate 5:5) in order to produce the expected compound (34 mg; 80% yield).

MS/LC: calculated MM=429.2; m/z=430.1 (MH+)

NMR (¹H, 400 MHz, DMSO-d₆): δ1.23-1.24 (2s, 12H), 1.65 (s, 4H), 2.93 (s, 3H), 2.97 (t, 2H), 4.41 (t, 2H), 7.14 (AB, 2H), 7.28 (AB, 2H), 7.45 (AB, 1H), 7.63 (AB, 1H), 7.82 (s, 1H), 9.62 (s, 1H).

In a manner analogous to the procedure described for 2-{4-[(methylsulphonyl)amino]phenyl}ethyl-5,5,8,8-tetramethyl-5,6,7,8-tetrahydronaphthalene-2-carboxylate, the following compounds were prepared:

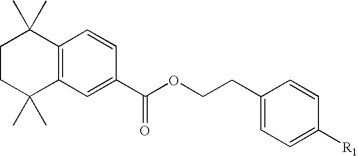

in which R₁ represents one of the radicals below:

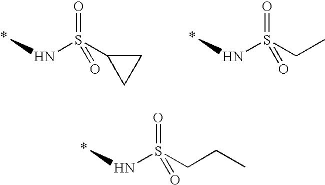

Preparation According to Reaction Diagram N

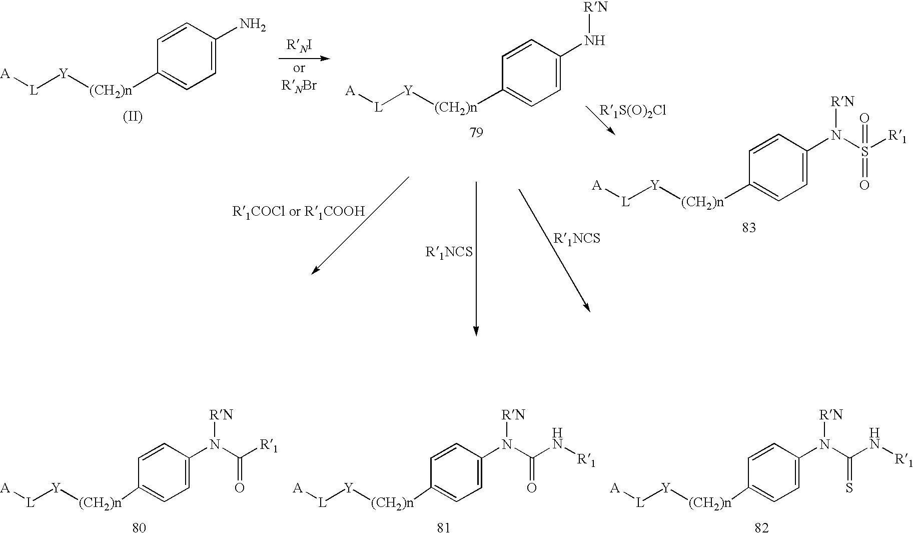

As described in Diagram N, the aniline of general formula (II) can react with a bromine or iodine derivative, in the presence of an organic or inorganic base, at a temperature of 18 to 150° C. in order to produce the alkylated the aniline (79). The aniline (79) can then be either coupled to an acid chloride in the presence of a tertiary base such as triethylamine or diisopropylethylamine at a temperature of approximately 0° C. to 25° C. for 30 min to 3 hours, or coupled to an acid in the presence of a coupling agent such as diisopropylcarbodiimide (DIC), dicyclohexylcarbodiimide (DCC), 1-(3-dimethylaminopropyl)-3-ethylcarbodiimide hydrochloride (EDC) or carbonyldiimidazole (CDI), with or without 1-hydroxybenzotriazole (HOBt), or with Mukaiyama's reagent (2-chloro-1-methyl-pyridinium chloride) in the presence of a tertiary base such as triethylamine or diisopropylethylamine, in an inert organic solvent such as methylene chloride, tetrahydrofuran or dimethylformamide at ambient temperature for 3 to 24 hours or alternatively heated under microwaves at a temperature of 80 to 120° C. (Biotage® equipment), in a sealed tube, for 10 to 30 minutes, in order to produce the corresponding amide (80). The aniline (79) can react with an isocyanate or an isothiocyanate in an inert organic solvent such as methylene chloride or tetrahydrofuran at ambient temperature in order to produce respectively the urea (81) and the thiourea (82). The aniline (79) can also react with a sulphonyl chloride, in an aprotic organic solvent such as dichloromethane or THF, in the presence of a tertiary base such as triethylamine, at a temperature of 0 to 60° C., for 1 to 24 hours, in order to produce the corresponding sulphonamide (83).

A subject of the invention is also a method for the preparation of a compound of formula (I) as defined above and in which R₁ represents the —NH—C(O)—R′₁ radical, characterized in that the aniline of formula (II):

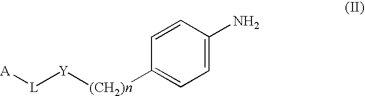

in which A, L, Y and n are as defined above, is coupled

either to an acid chloride of formula R′₁COCl in which R′₁ is as defined above, in the presence of a tertiary base, in an inert organic solvent, at a temperature comprised between 0° C. and ambient temperature for 30 min to 3 hours,

or to an acid of formula R′₁COOH in which R′₁ is as defined above, in the presence either of a coupling agent or of Mukaiyama's reagent, in the presence of a tertiary base, in an inert organic solvent,

in order to produce the corresponding amide

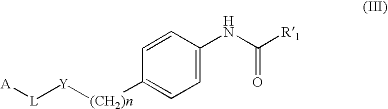

A subject of the invention is also a method for the preparation of a compound of formula (I) as defined above and in which R₁ represents the —NH—C(Z)-NHR′₁ radical, characterized in that the aniline of formula (II)

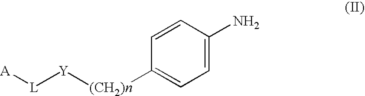

in which A, L, Y and n are as defined above, is reacted with an isocyanate or an isothiocyanate of formula R′₁N═C═Z in which R′₁ and Z are as defined above, in an inert organic solvent at a temperature comprised between ambient temperature and 60° C., in order to produce the corresponding urea or thiourea of formula (IV)

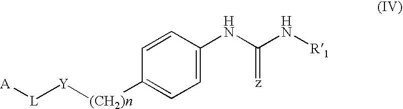

A subject of the invention is also a method for the preparation of a compound of formula (I) as defined above and in which R₁ represents the —N═C(NH₂)R′₁ radical, characterized in that the aniline of formula (II):

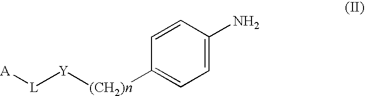

in which A, L, Y and n are as defined above, is treated with a thioimidate of formula NH═C(SMe)R′₁ in which R′₁ is as defined above, in a polar solvent, at a temperature comprised between ambient temperature and 80° C. for 2 to 24 hours, in order to produce the amidine of formula (V)

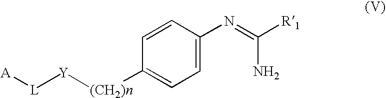

A subject of the invention is also a method for the preparation of a compound of formula (I) as defined above and in which R₁ represents the —NH—S(O)₂—R′₁ radical, characterized in that the aniline of formula (II)

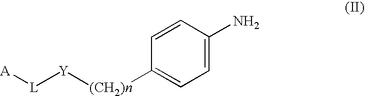

in which A, L, Y and n are as defined above, is reacted with a sulphonyl chloride of formula R′₁S(O)₂Cl in which R′₁ is as defined above, in an aprotic organic solvent in the presence of a tertiary base, at a temperature of 0 to 60° C. for 1 to 24 hours, in order to produce the corresponding sulphonamide (VI)

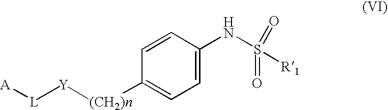

The compounds of the present invention possess useful pharmacological properties. It has thus been discovered that the compounds of the present invention have a good affinity for certain sub-types of cannabinoid receptors, in particular the CB2 receptors. They are particularly useful for treating the pathological states and the diseases in which one or more cannabinoid receptors are involved.

The compounds of the present invention can thus be used in different therapeutic applications. They can advantageously be used for the treatment and prevention of the pathological states and diseases associated with the activity of the cannabinoid receptors such as cell proliferation disorders such as cancer, immune disorders, inflammation, pain, osteoporosis, atherosclerosis, epilepsy, nausea associated with chemotherapy treatments, fibrosis, gastro-intestinal disorders, neurodegenerative diseases including multiple sclerosis and dyskinesia, Parkinson's disease, Huntington's chorea, Alzheimer's disease. They can also be used in order to prevent or cure diseases associated with motor function such as Tourette's syndrome, or in order to provide neuroprotection. The compounds according to the present invention can be administered alone or in combination with other agents linked to treatments of the symptoms or the cause of the disease or pathological state as mentioned above. An illustration of the pharmacological properties of the compounds of the invention will be found hereafter, in the experimental part.

A subject of the present application is also pharmaceutical compositions containing, as active ingredient, at least one product of formula I as defined above, or an addition salt with the pharmaceutically acceptable mineral or organic acids of said product of formula I, in combination with a pharmaceutically acceptable support.

A subject of the present application is also the use of the compounds according to the present invention, for the preparation of a medicament for the treatment of cell proliferation disorders and preferably for the treatment of cancer.

A subject of the present application is also the use of the compounds according to the present invention, for the preparation of a medicament for the treatment of immune disorders, inflammation, pain, osteoporosis, fibrosis, gastro-intestinal disorders, neurodegenerative diseases including multiple sclerosis and dyskinesia, Parkinson's disease.

The pharmaceutical composition can be in the form of a solid, for example, powders, granules, tablets, gelatin capsules or suppositories. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax.

The pharmaceutical compositions containing a compound of the invention can also be presented in liquid form, for example, solutions, emulsions, suspensions or syrups. Appropriate liquid supports can be, for example, water, organic solvents such as glycerol or the glycols, as well as their mixtures, in varying proportions, in water, added to pharmaceutically acceptable oils or fats. The sterile liquid compositions can be used for intramuscular, intraperitoneal or sub-cutaneous injections and the sterile compositions can also be administered intravenously.

All the technical and scientific terms used in the present text have the meaning known to a person skilled in the art. Moreover, all the patents (or patent applications) as well as the other bibliographical references are incorporated by way of reference.

### Experimental Part:

The compounds according to the invention obtained according to the previously described procedures are shown in the table below.

The compounds are characterized by their retention time (rt) and their molecular peak determined by mass spectrometry (MH+).

For the mass spectrometry, a single quadripole mass spectrometer (Micromass, Platform model) equipped with an electrospray source is used with a resolution of 0.8 Da at 50% valley. Calibration is carried out monthly between the masses 80 and 1000 Da using a calibration mixture of sodium iodide and rubidium iodide in solution in an isopropanol/water mixture (1/1 Vol.).

For the liquid chromatography, a Waters system comprising an in-line degasser, a Waters 600 quaternary pump, a Gilson 233 plate sampling injector and a Waters PAD 996 UV detector is used.

The elution conditions used are as follows:

Eluent: A water + 0.02% trifluoroacetic acid; B acetonitrile

| T (min) | A % | B % |
|---|---|---|
| 1 | 95 | 5 |
| 8.5 | 5 | 95 |
| 10.5 | 5 | 95 |
| 10.6 | 95 | 5 |
| 14.9 | 95 | 5 |
| 15.0 | 95 | 5 |

Flow rate: 1 ml/min; Injection: 10 μl; Column: Uptisphere ODS 3 μm 75*4.6 mm i.d.

These examples are presented in order to illustrate the above procedures and should in no event be considered as limiting the scope of the invention.

| Examples | Molecular structures | [M + H]+ | rt (min) |
|---|---|---|---|
| 1 | 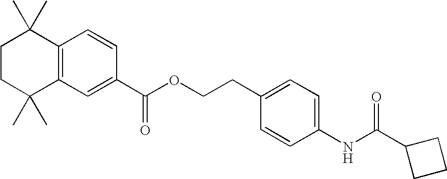 | 434.3 | 11.7 |
| 2 | 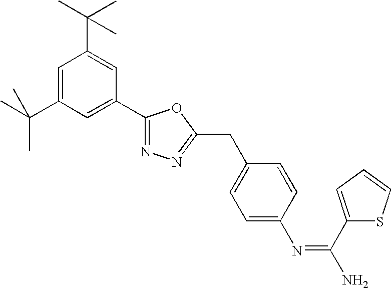 | 473.3 | 9.7 |
| 3 | 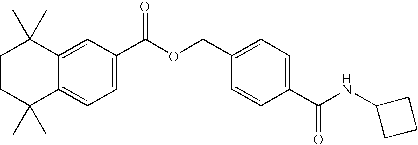 | 420.3 | 11.5 |
| 4 | 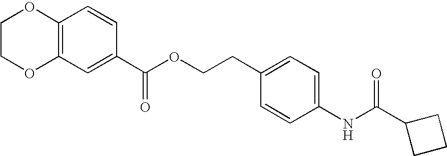 | 382.2 | 10.8 |
| 5 | 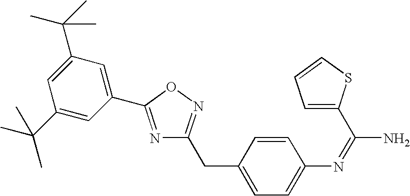 | 473.3 | 9.1 |
| 6 | 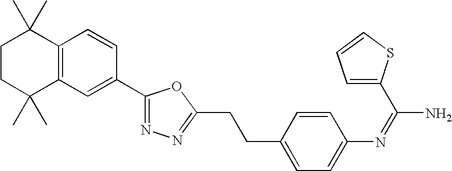 | 485.3 | 8.7 |
| 7 | 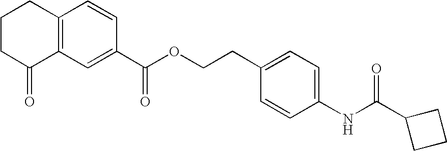 | 392.2 | 9.7 |
| 8 | 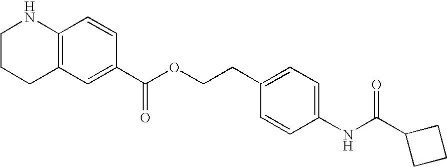 | 374.2 | 9.6 |
| 9 | 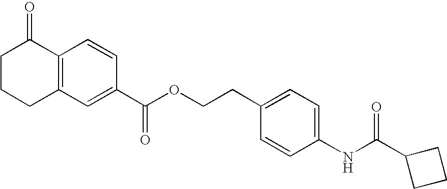 | 392.2 | 9.8 |
| 10 | 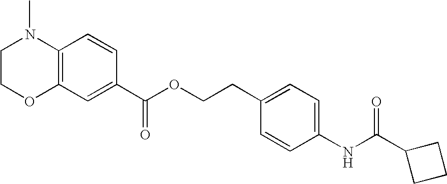 | 395.2 | 9.7 |
| 11 | 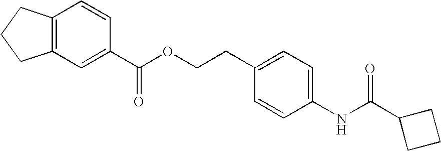 | 364.3 | 10.6 |
| 12 | 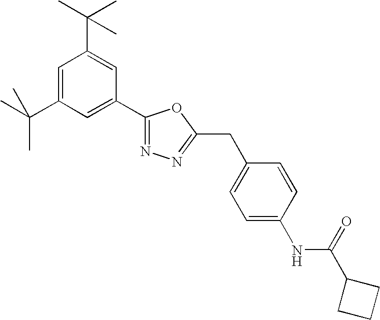 | 446.3 | 11.3 |
| 13 | 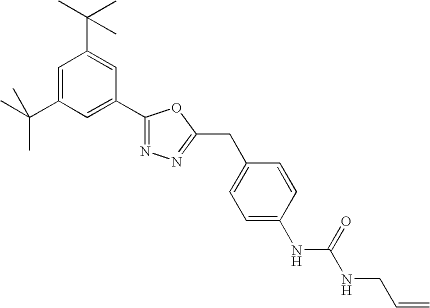 | 447.3 | 10.7 |
| 14 | 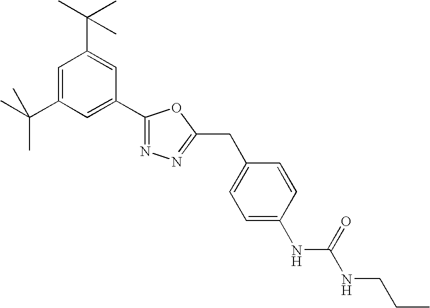 | 449.4 | 10.9 |
| 15 | 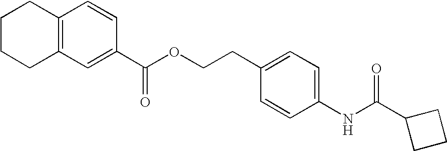 | 378.3 | 11.0 |
| 16 | 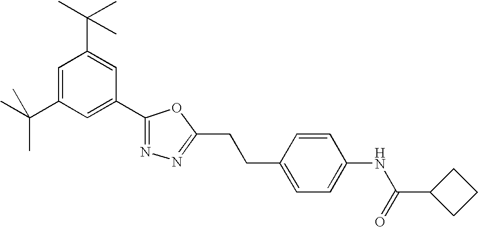 | 460.4 | 11.4 |
| 17 | 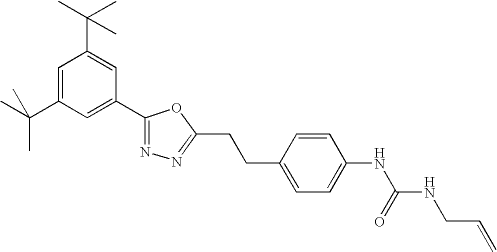 | 461.4 | 10.9 |
| 18 | 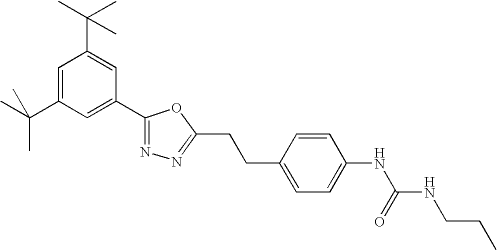 | 463.4 | 11.0 |
| 19 | 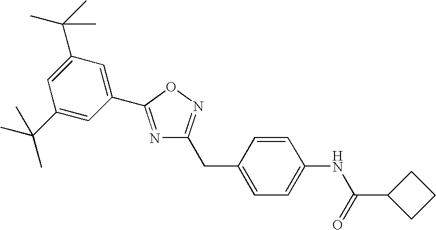 | 446.3 | 11.9 |
| 20 | 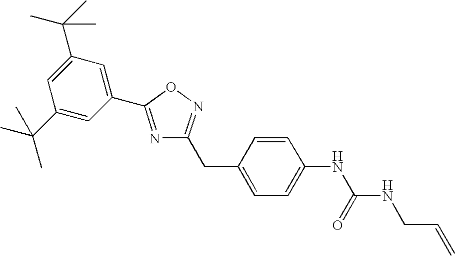 | 447.4 | 11.6 |
| 21 | 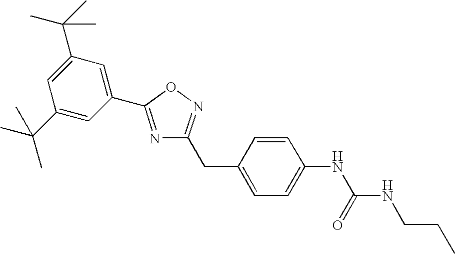 | 449.4 | 11.7 |
| 22 | 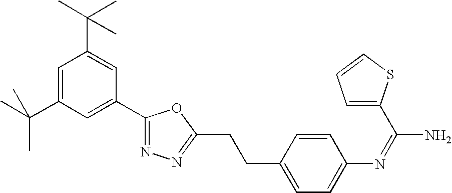 | 487.4 | 8.8 |
| 23 | 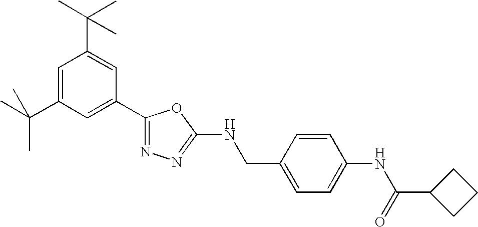 | 461.4 | 10.6 |
| 24 | 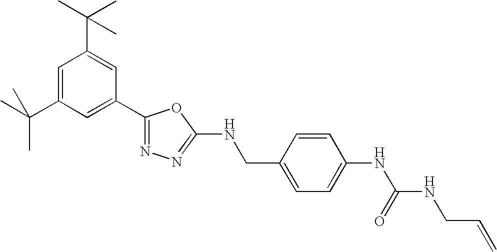 | 462.3 | 10.1 |
| 25 | 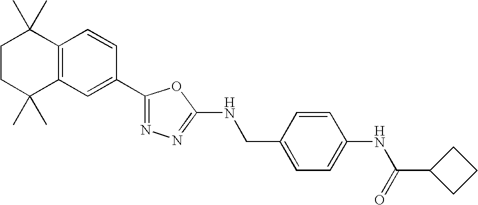 | 459.3 | 10.5 |
| 26 | 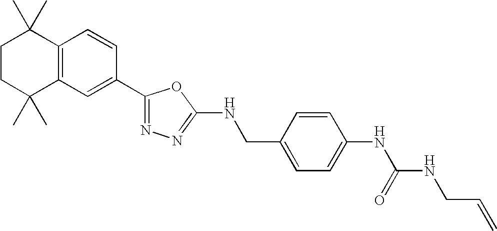 | 460.3 | 10.0 |
| 27 | 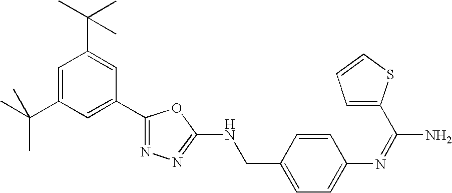 | 488.3 | 9.4 |
| 28 | 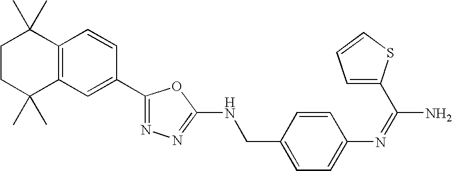 | 486.3 | 9.3 |
| 29 | 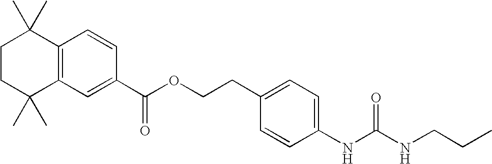 | 437.3 | 11.3 |
| 30 | 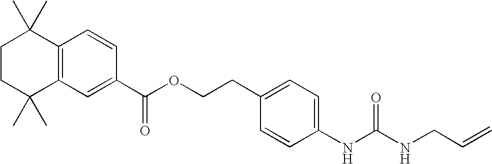 | 435.3 | 11.2 |
| 31 | 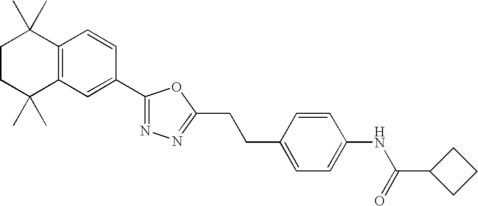 | 458.3 | 11.3 |
| 32 | 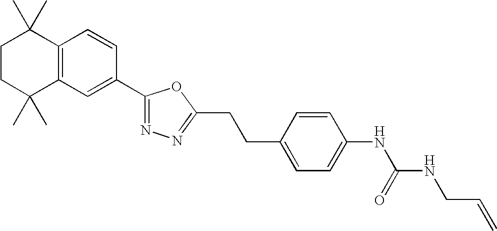 | 459.3 | 10.8 |
| 33 | 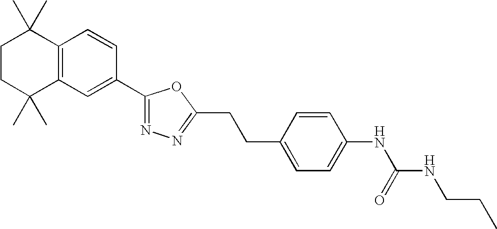 | 461.3 | 10.8 |
| 34 | 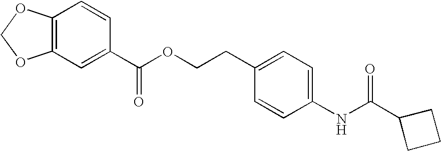 | 368.2 | 9.7 |
| 35 | 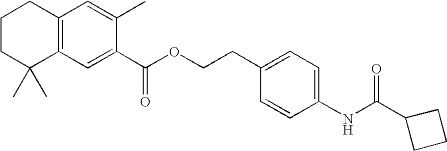 | 420.3 | 11.7 |
| 36 | 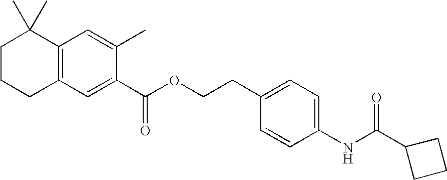 | 420.3 | 11.8 |
| 37 | 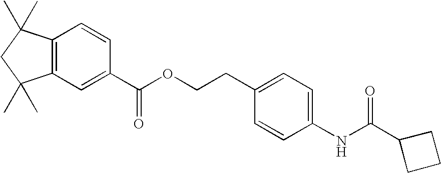 | 420.3 | 11.5 |
| 38 | 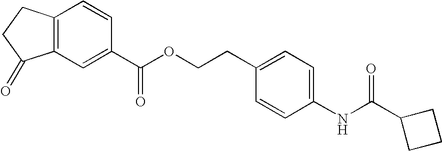 | 378.3 | 9.2 |
| 39 | 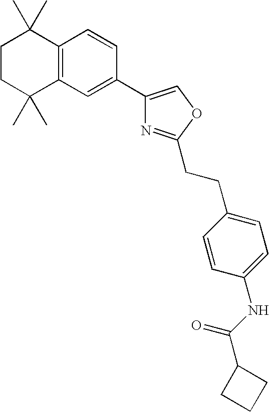 | 457.4 | 11.7 |
| 40 | 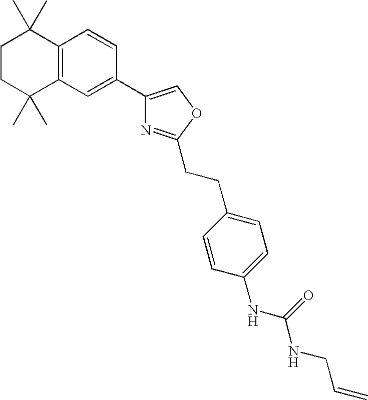 | 458.3 | 11.1 |
| 41 | 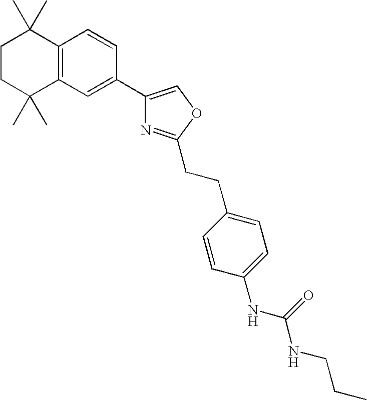 | 460.4 | 11.2 |
| 42 | 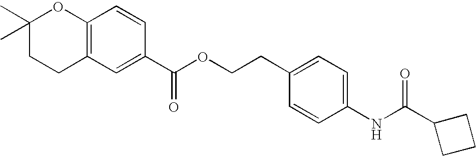 | 408.2 | 10.6 |
| 43 | 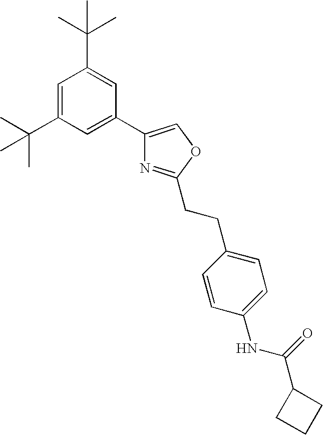 | 460.4 | 11.2 |
| 44 | 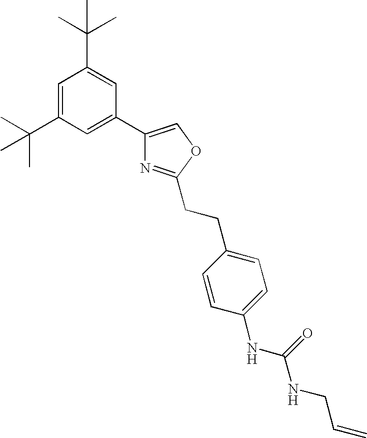 | 459.4 | 11.8 |
| 45 | 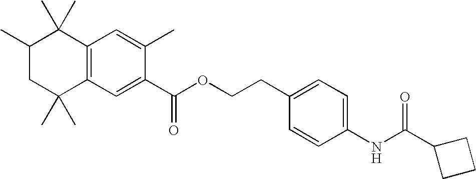 | 462.3 | 11.4 |
| 46 | 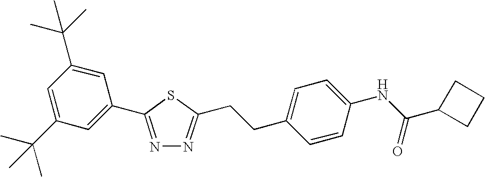 | 476.3 | 10.9 |
| 47 | 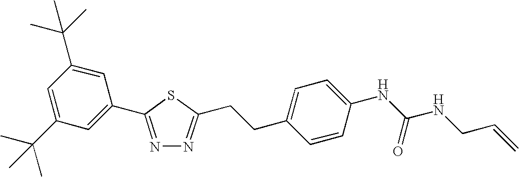 | 477.3 | 10.5 |
| 48 | 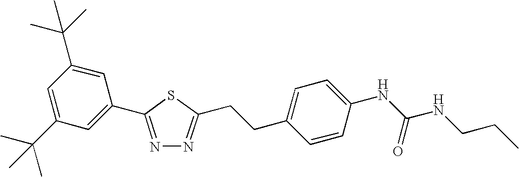 | 479.3 | 10.6 |
| 49 | 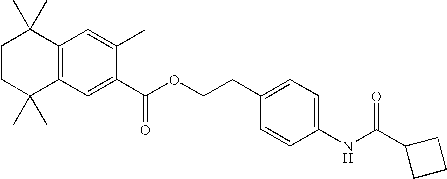 | 448.2 | 11.6 |
| 50 | 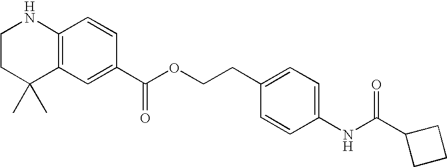 | 407.2 | 9.8 |
| 51 | 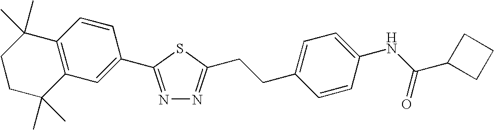 | 474.3 | 12.5 |
| 52 | 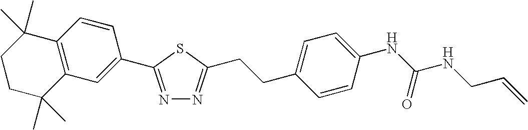 | 475.2 | 11.9 |
| 53 | 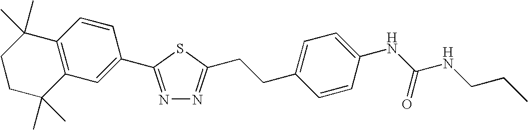 | 477.3 | 12.1 |
| 54 | 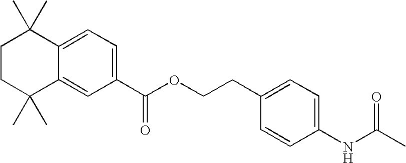 | 394.2 | 10.9 |
| 55 | 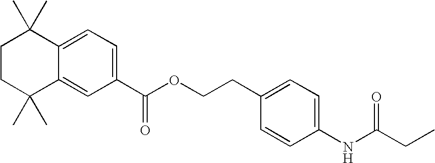 | 408.2 | 11.1 |
| 56 | 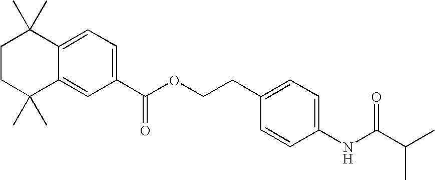 | 422.2 | 11.3 |
| 57 | 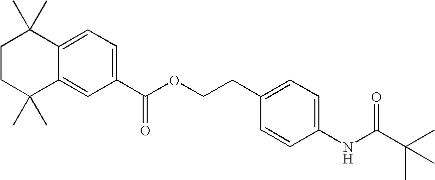 | 436.2 | 11.6 |
| 58 | 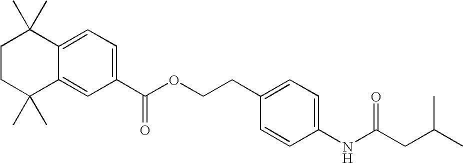 | 436.2 | 11.5 |
| 59 | 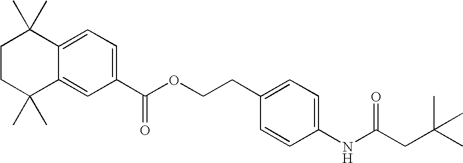 | 450.2 | 11.7 |
| 60 | 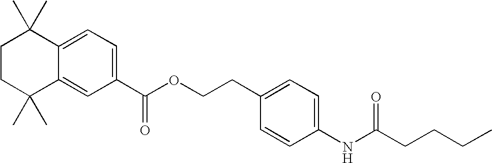 | 436.2 | 11.5 |
| 61 | 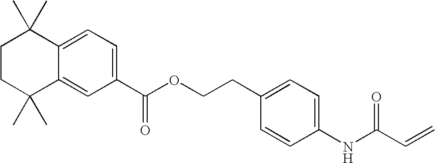 | 406.2 | 11.1 |
| 62 | 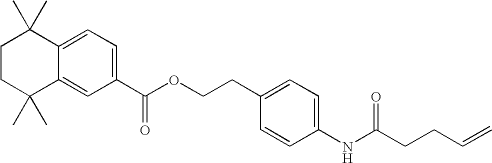 | 434.2 | 11.4 |
| 63 | 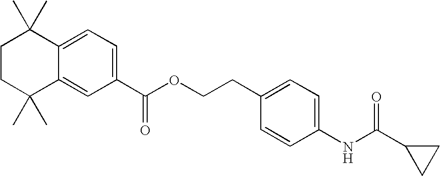 | 420.2 | 11.2 |
| 64 | 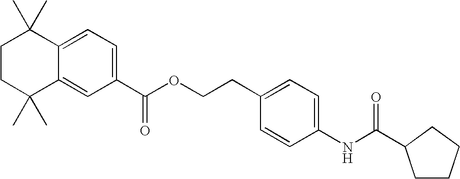 | 448.2 | 11.6 |
| 65 | 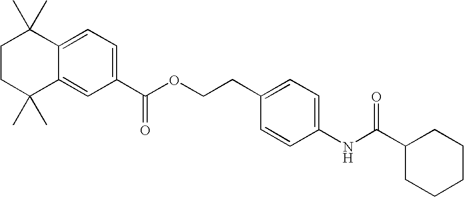 | 462.3 | 11.8 |
| 66 | 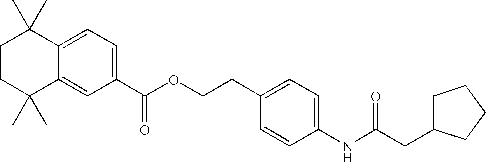 | 462.3 | 11.8 |
| 67 | 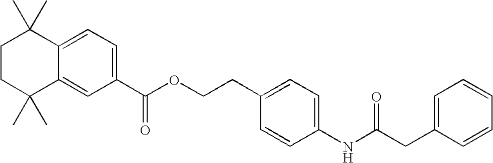 | 470.2 | 11.4 |
| 68 | 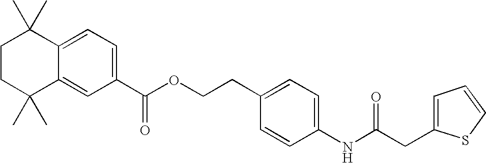 | 476.2 | 11.4 |
| 69 | 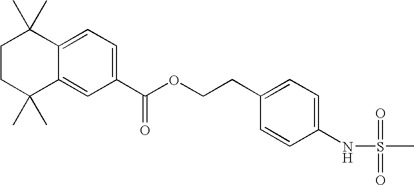 | 430.1 | 11.0 |
| 70 | 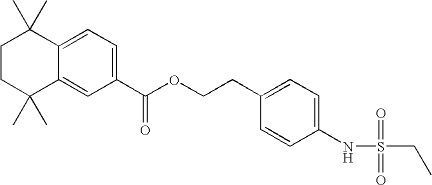 | 444.2 | 11.1 |
| 71 | 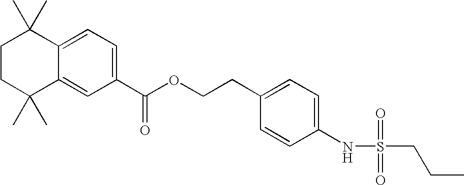 | 458.2 | 11.3 |
| 72 | 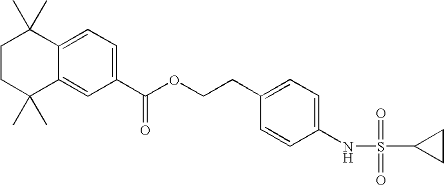 | 456.2 | 11.2 |
| 73 | 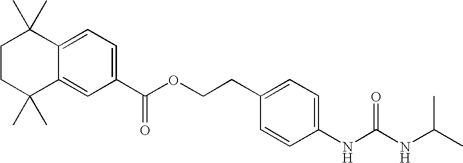 | 437.4 | 12.2 |
| 74 | 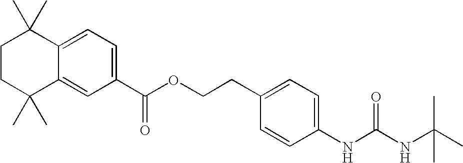 | 451.4 | 12.7 |
| 75 | 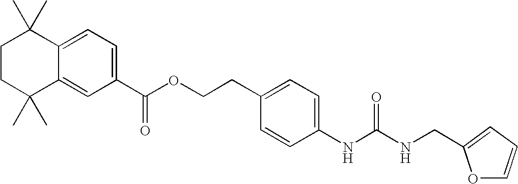 | 475.4 | 12.2 |
| 76 | 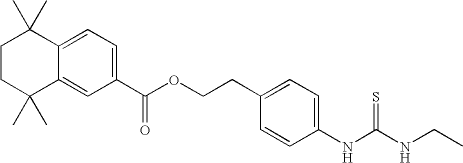 | 439.3 | 11.1 |
| 77 | 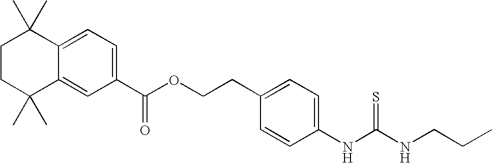 | 453.4 | 11.3 |
| 78 | 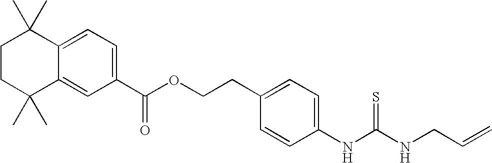 | 451.4 | 11.2 |
| 79 | 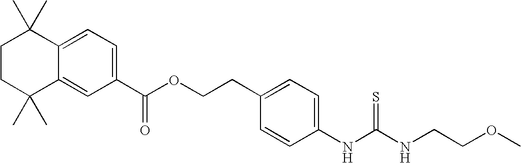 | 469.4 | 11.1 |
| 80 | 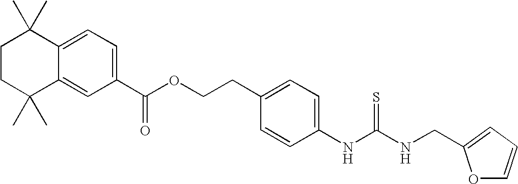 | 491.4 | 11.2 |
| 81 | 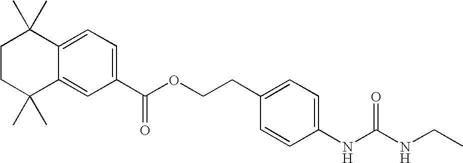 | 423.4 | 10.9 |
| 82 | 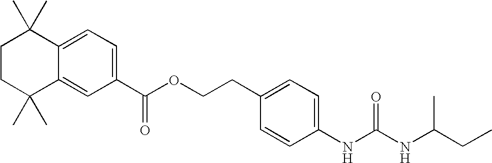 | 451.4 | 11.2 |
| 83 | 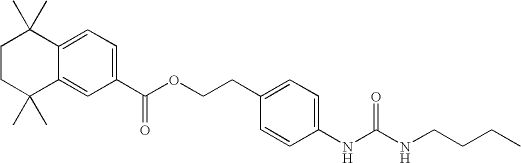 | 451.4 | 11.3 |
| 84 | 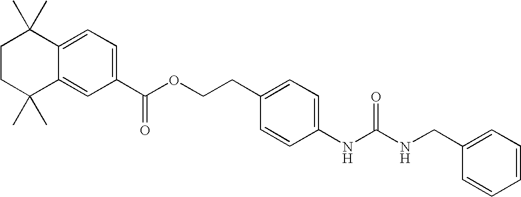 | 485.4 | 11.3 |
| 85 | 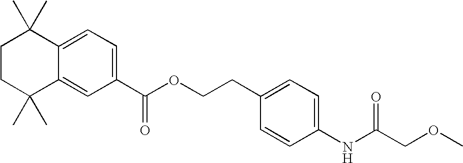 | 424.3 | 11.1 |
| 86 | 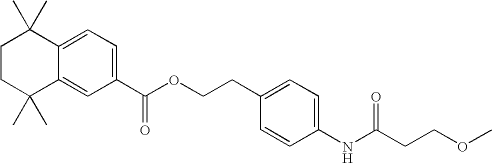 | 438.3 | 11.0 |
| 87 | 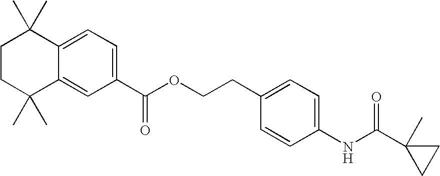 | 434.4 | 11.4 |
| 88 | 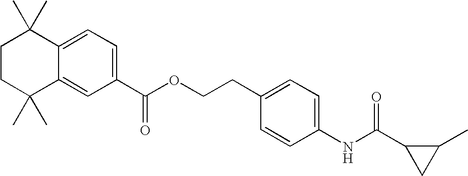 | 434.4 | 11.4 |
| 89 | 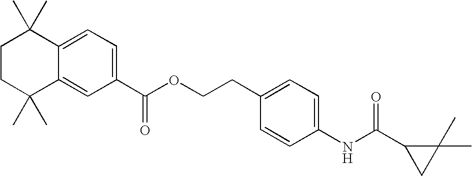 | 448.4 | 11.7 |
| 90 | 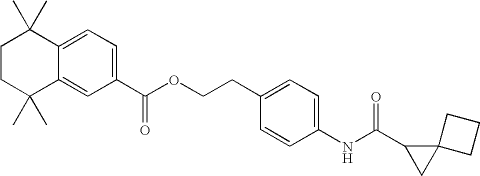 | 460.4 | 11.7 |
| 91 | 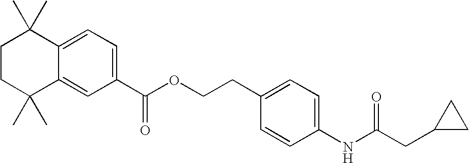 | 434.4 | 11.3 |
| 92 | 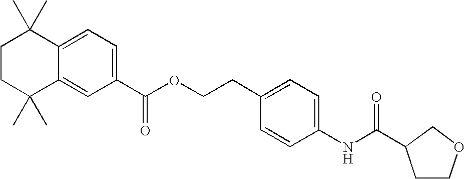 | 450.4 | 10.9 |
| 93 | 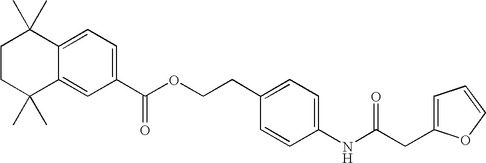 | 460.4 | 11.2 |
| 94 | 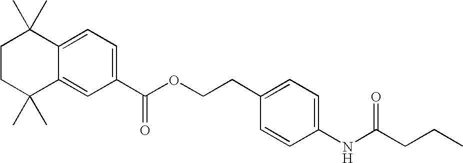 | 422.4 | 11.3 |
| 95 | 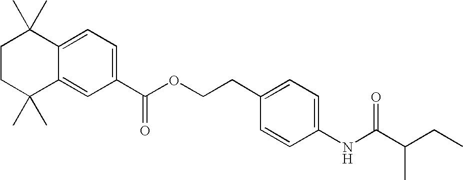 | 436.4 | 11.5 |
| 96 | 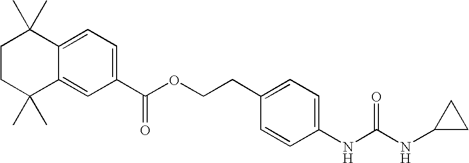 | 435.4 | 10.8 |
| 97 | 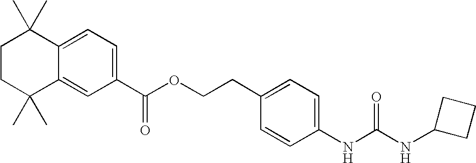 | 449.4 | 11.1 |
| 98 | 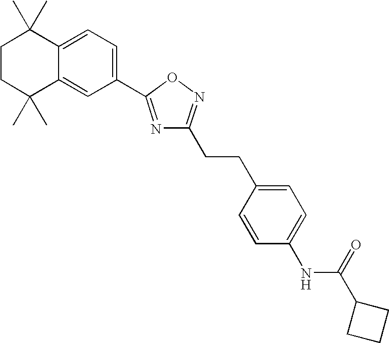 | 458.2 | 11.7 |

### Pharmacological Study

The affinity of the compounds of the present invention for the different sub-types of cannabinoid receptors was measured according to procedures analogous to those described hereafter for the human CB2 receptor.

### Study of the Affinity of the Compounds for the Human CB2 Cannabinoid Receptors:

The affinity of the compounds of the invention for the human CB2 receptors is determined by measuring the inhibition of the binding of [³H]-CP55940 to transfected CHO-K1 cell membrane preparations.

The CHO-K1 cells expressing the human CB2 receptors in stable fashion are cultured in an RPMI 1640 medium containing 10% foetal calf serum, 2 mM of glutamine, 100 U/ml of penicillin, 0.1 mg/ml of streptomycin and 0.5 mg/ml of G418. The cells are collected with 0.5 mM of EDTA and centrifuged at 500 g for 5 minutes at 4° C. The pellet is re-suspended in phosphate buffered saline medium (PBS) and centrifuged at 500 g for 5 minutes at 4° C. The pellet is re-suspended in a Tris 50 mM buffer medium at pH 7.4 and centrifuged at 500 g for 5 minutes at 4° C. The cells are lysed by sonication and centrifuged at 39,000 g for 10 minutes at 4° C. The pellet is re-suspended in the Tris 50 mM buffer medium at pH 7.4 and centrifuged at 50,000 g for 10 minutes at 4° C. The membranes obtained in this last pellet are stored at −80° C.

Measurement of the competitive inhibition of the binding of the [³H]-CP55940 to the CB2 receptors is carried out in duplicate using 96-well polypropylene plates. The cell membranes (10 μg of proteins/well) are incubated with the [³H]-CP55940 (1 nM) for 60 minutes at 25° C. in a Tris-HCl 50 mM buffer medium, pH 7.4, comprising 0.1% bovine serum albumin (BSA), 5 mM of MgCl₂, and 50 μg/ml of bacitracin.

The bound [³H]-CP55940 is separated from the free [3H]-CP55940 by filtration through GF/C glass fibre filter plates (Unifilter, Packard) pre-impregnated with 0.1% polyethylenimine (P.E.I.), using a Filtermate 196 (Packard). The filters are washed with Tris-HCl 50 mM buffer, pH 7.4 at 0-4° C. and the radioactivity present is determined using a counter (Packard Top Count).

The specific binding is obtained by subtracting the non-specific binding (determined in the presence of 0.1 μM of WIN55212-2 from the total binding). The data are analyzed by computer-assisted non-linear regression (MDL). For each test, a Cheng-Prusoff correction is made in order to convert the IC50 to the inhibition constant, Ki.

Thus,

$$Ki = \frac{IC 50}{1 + [ L ] / Kd}$$

where [L] is the concentration of the radioligand used in the test and Kd is the radioligand equilibrium disassociation constant.

The ranges of values of the inhibition constant thus measured are presented in the table below (the existence of a constant Ki less than 5 μM or 0.5 μM is represented by the presence of “x”).

| Examples | Ki < 5 μM | Ki < 0.5 μM |
|---|---|---|
| 1 | x | x |
| 2 | x | x |
| 3 | x | x |
| 4 | x | |
| 5 | x | x |
| 6 | x | |
| 7 | x | |
| 8 | x | x |
| 9 | | |
| 10 | x | x |
| 11 | x | x |
| 12 | x | x |
| 13 | x | x |
| 14 | x | x |
| 15 | x | x |
| 16 | x | x |
| 17 | x | x |
| 18 | x | x |
| 19 | x | x |
| 20 | x | x |
| 21 | x | x |
| 22 | x | x |
| 23 | x | |
| 24 | x | x |
| 25 | x | x |
| 26 | x | x |
| 27 | x | x |
| 28 | x | |
| 29 | x | x |
| 30 | x | x |
| 31 | x | x |
| 32 | x | x |
| 33 | x | x |
| 34 | x | |
| 35 | x | x |
| 36 | x | x |
| 37 | x | x |
| 38 | x | |
| 39 | x | x |
| 40 | x | x |
| 41 | x | x |
| 42 | x | x |
| 43 | x | x |
| 44 | x | x |
| 45 | x | x |
| 46 | x | x |
| 47 | x | x |
| 48 | x | x |
| 49 | x | x |
| 50 | x | x |
| 51 | x | x |
| 52 | x | x |
| 53 | x | x |
| 54 | x | x |
| 55 | x | x |
| 56 | x | x |
| 57 | x | x |
| 58 | x | x |
| 59 | x | x |
| 60 | x | x |
| 61 | x | x |
| 62 | x | x |
| 63 | x | x |
| 64 | x | x |
| 65 | x | x |
| 66 | x | x |
| 67 | x | x |
| 68 | x | x |
| 69 | x | x |
| 70 | x | x |
| 71 | x | x |
| 72 | x | x |
| 73 | x | x |
| 74 | x | x |
| 75 | x | x |
| 76 | x | x |
| 77 | x | x |
| 78 | x | x |
| 79 | x | x |
| 80 | x | x |
| 81 | x | x |
| 82 | x | x |
| 83 | x | x |
| 84 | x | x |
| 85 | x | x |
| 86 | x | x |
| 87 | x | x |
| 88 | x | x |
| 89 | x | x |
| 90 | x | x |
| 91 | x | x |
| 92 | x | x |
| 93 | x | x |
| 94 | x | x |
| 95 | x | x |
| 96 | x | x |
| 97 | x | x |
| 98 | x | x |

The agonistic or antagonistic activity of the CB2 receptors of the compounds of the present invention was determined by measuring the production of cyclic AMP by the CHO-K1 cells transfected by the CB2 receptor.

### Measurement of the Production of Intracellular Cyclic AMP Via the CB2 Receptors:

The CHO-K1 cells expressing the CB2 cannabinoid receptors are cultured in 384-well plates in an RPMI 1640 medium with 10% foetal calf serum and 0.5 mg/ml of G418. The cells are washed twice with 50 μl of RPMI medium comprising 0.2% BSA and 0.5 mM of 3-isobutyl-1-methylxanthine (IBMX).

In order to measure the agonistic effect of a compound, the cells are incubated for 5 minutes at 37° C. in the presence of 0.5 mM of IBMX, then the stimulation of the production of cyclic AMP is obtained by adding 5 μM of Forskolin then the inhibition is measured by the addition of the compound at concentrations comprised between 1 μM and 10 μM in duplicate for 20 minutes at 37° C. The antagonistic effect of a compound is measured by inhibiting the inhibition of the production of cyclic AMP induced by WIN55212-2 in the presence of 5 μM of Forskolin, at concentrations comprised between 1 pM and 10 μM, in the presence of the compound to be tested, at concentrations comprised between 1 nM and 10 μM, in duplicate for 20 minutes at 37° C.

The reaction medium is eliminated and 80 μl of lysis buffer is added. The level of intracellular cyclic AMP is measured by a competition test with fluorescent cyclic AMP (CatchPoint, Molecular Devices).

1. A compound of formula (I) 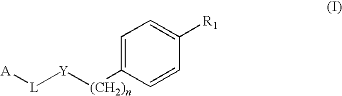
in racemic or enantiomeric form or any combinations of these forms and in which
A represents an A₁ or A₂ radical, wherein A₁ and A₂ are 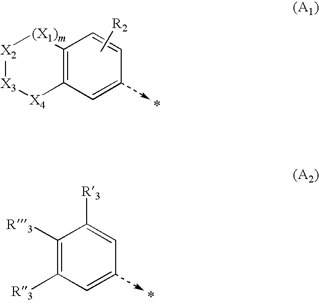
and X₁, X₂, X₃ and X₄ represent, independently, an oxygen or sulfur atom, or a radical of formula —NR_{N}— or —C(R₄R₅)— with the proviso that the chain —(X₁)ₘ-X₂-X₃-X₄- does not contain adjacent heteroatoms;
m represents 0 or 1;
R₄ and R₅ represent, independently, a hydrogen atom or a (C₁-C₆)alkyl radical optionally substituted by one or more identical or different halos, or R₄ and R₅ together form the oxo radical;
R_{N} represents a hydrogen atom, a (C₁-C₆)alkyl radical or (C₁-C₆)alkyl-carbonyl radical;
R₂ represents a hydrogen atom or a (C₁-C₆)alkyl radical;
R′₃, R″₃ and R′″₃ represent, independently, a hydrogen atom, a hydroxy, (C₁-C₆)alkyl or (C₁-C₄)alkoxy radical;
L represents
—C(O)—O— or a radical corresponding to an oxadiazole, oxazole or thiadiazole ring if A represents the (A₁) radical, or
a radical corresponding to an oxadiazole, oxazole or thiadiazole ring if A represents the (A₂) radical;
Y represents a covalent bond or a —NH— radical;
n represents 1, 2 or 3;
R₁ represents a —NR′_{N}—C(O)—R′₁, —NR′_{N}—S(O)₂—R′₁, —NR′_{N}—C(Z)-NHR′₁, —C(O)—NH—R′₁, or —N═C(NH₂)R′₁, radical;
R′_{N} represents a hydrogen atom or a (C₁-C₄)alkyl radical;
Z represents a sulfur or oxygen atom;
R′₁ represents a (C₁-C₈)alkyl, (C₂-C₆)alkenyl, (C₁-C₈)hydroxyalkyl, (C₃-C₇)cycloalkyl, spiro-cycloalkyl, (C₃-C₇)heterocycloalkyl, heteroaryl, (C₁-C₈)alkoxy, (C₁-C₈)alkoxy-(C₁-C₈)alkyl radical, or a (CH₂)ₚ—R′₂ radical, wherein the radical is optionally substituted by one or more identical or different substituents including halo, (C₁-C₆)alkyl, or (C₁-C₆)haloalkyl;
p represents 1, 2 or 3; and
R′₂ represents a (C₃-C₇)cycloalkyl, (C₃-C₇)heterocycloalkyl, aryl or heteroaryl radical, wherein the radical is optionally substituted by one or more identical or different substituents including halo, (C₃-C₆)alkyl, (C₁-C₆)alkoxy or (C₁-C₆)haloalkyl;
with the proviso that
i) when L represents —C(O)—O—, then Y represents a covalent bond;
ii) at least one of the R′₃, R″₃ or R′″₃ radicals is not a hydrogen atom;
iii) when R′″₃ represents a hydroxy or (C₁-C₄)alkoxy radical, then R₁ represents a —NR′_{N}—C(O)—R′₁, —NR′_{N}—S(O)₂—R′₁ or —NR′_{N}—C(Z)-NHR′₁ radical;
or a pharmaceutically acceptable salt thereof.

2. The compounds of claim 1, wherein A represents the (A₁) radical.

3. The compounds of claim 1, wherein
X₁, X₂, X₃ and X₄ represent, independently, an oxygen atom, or a radical of formula —NR_{N}— or —C(R₄R₅)—;
R_{N} represents a hydrogen atom or a (C₁-C₆)alkyl radical;
R₄ and R₅ represent, independently, a hydrogen atom or a (C₁-C₆)alkyl radical, or together form an oxo radical; and m represents 0 or 1.

4. The compound of claim 1, wherein
X₁ represents an oxygen atom or a radical of formula —NR_{N}— or —C(R₄R₅)—;
X₂ and X₃ represent, independently, an oxygen atom or a radical of formula —C(R₄R₅)—;
X₄ represents an oxygen atom or —C(R₄R₅)—; and
m represents 0 or 1.

5. The compound of claim 4, wherein
X₁ represents a radical of formula —NR_{N}— or —C(R₄R₅)—;
X₂ and X₃ represent, independently, a radical of formula —C(R₄R₅)—;
X₄ represents an oxygen atom or —C(R₄R₅)—;
R₄ and R₅ represent, independently, a hydrogen atom or a methyl radical;
R_{N} represents a hydrogen atom or a methyl radical; and
m represents 0 or 1.

6. The compound of claim 1, wherein A represents the (A₂) radical.

7. The compound of claim 1, wherein R′₃, R″₃ and R′″₃ represent, independently, a hydrogen atom or a (C₁-C₆)alkyl radical.

8. The compound of claim 1, wherein R′₃ and R″₃ represent, independently, a tert-butyl radical, and R′″₃ represents a hydrogen atom.

9. The compound of claim 1, wherein n represents 1 or 2.

10. The compound of claim 1, wherein L represents —C—O—.

11. The compound of claim 1, wherein L represents a radical corresponding to an oxadiazole ring.

12. The compound of claim 1, wherein L represents a radical corresponding to an oxazole ring.

13. The compound of claim 1, wherein L represent a radical corresponding to a thiadiazole ring.

14. The compound of claim 1, wherein Y represents a covalent bond and n represents 2.

15. The compound of claim 1, wherein
R₁ represents a —NR′_{N}—C(O)—R′₁, —NR′_{N} —S(O)₂—R′₁, —NR′_{N}—C(Z)-NHR′₁, —C(O)—NH—R′₁ or —N═C(NH₂)R′₁, radical;
R′_{N} represents a hydrogen atom;
Z represents a sulfur or oxygen atom;
R′₁ represents a (C₁-C₈)alkyl, (C₂-C₆)alkenyl, (C₃-C₇)cycloalkyl, spiro-cycloalkyl, (C₃-C₇)heterocycloalkyl, heteroaryl, (C₁-C₈)alkoxy-(C₁-C₈)alkyl radical, or a (CH₂)ₚ—R′₂ radical, wherein the radical is optionally substituted by one or more identical or different (C₁-C₆)alkyl substituents; and
R′₂ represents a (C₃-C₇)cycloalkyl, (C₃-C₇)heterocycloalkyl, aryl or heteroaryl radical.

16. The compound of claim 1, wherein R₁ represents a —NR′_{N}—C(O)—R′₁, —NR′_{N}—S(O)₂—R′1, or —NR′_{N}—C(Z)-NHR′₁ radical, and R′_{N} represents a hydrogen atom.

17. The compound of claim 1, wherein
R₁ represents a —NR′_{N}—C(O)—R′₁ or —NR′_{N}—C(Z)-NHR′₁ radical and R′_{N} represents a hydrogen atom;
Z represents an oxygen atom;
R′₁ represents a (C₁-C₈)alkyl, (C₂-C₆)alkenyl, (C₃-C₇)cycloalkyl radical optionally substituted by one or more identical or different (C₁-C₆)alkyl substituents, spiro-cycloalkyl or a (CH₂)ₚ—R′₂ radical wherein p is 1; and
R′₂ represents a (C₃-C₇)cycloalkyl or heteroaryl radical.

18. The compound of claim 17, wherein
R₁ represents a —NR′_{N}—C(O)—R′₁ radical, and R′^{N} represents a hydrogen atom;
R′₁ represents a (C₁-C₆)alkyl, (C₂-C₆)alkenyl or (C₃-C₇)cycloalkyl radical chosen from cyclopropyl, cyclobutyl and cyclopentyl and wherein the radical is optionally substituted by one or more identical or different (C₁-C₆)alkyl substituents, spiro[2:3]hexane, or a (CH₂)ₚ—R′₂ radical wherein p is 1; and
R′₂ represents a (C₃-C₇)cycloalkyl radical chosen from cyclopropyl, cyclobutyl and cyclopentyl.

19. The compound of claim 17, wherein R₁ represents a —NR′_{N}—C(Z)-NHR′₁ radical;
R′^{N} represents a hydrogen atom and Z represents an oxygen atom; and
R′₁ represents a (C₁-C₆)alkyl or (C₂-C₆)alkenyl radical.

20. A method for the preparation of a compound of formula (III) 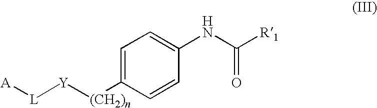
as defined in claim 1 and in which R₁ represents the —NH—C(O)—R′₁ radical, comprising the step of reacting the aniline of formula (II) 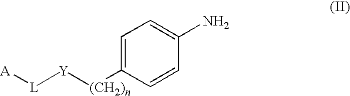
in which A, L, Y and n are as defined in claim 1, with
either an acid chloride of formula R′₁COCl in which R′₁ is as defined in claim 4, in the presence of a tertiary base in an inert organic solvent at a temperature between 0° C. and ambient temperature for 30 minutes to 3 hours,
or an acid of formula R′₁COOH in which R′₁ is as defined in claim 1, in the presence either of a coupling agent or of Mukaiyama's reagent, in the presence of a tertiary base, in an inert organic solvent.

21. A method for the preparation of a compound of formula (IV) 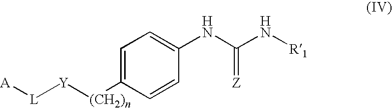
as defined in claim 1 and in which R₁ represents a —NH—C(Z)-NHR′ radical, comprising the step of reacting the aniline of formula (II) 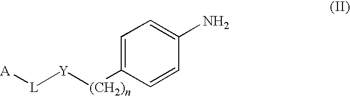
in which A, L, Y and n are as defined in claim 1, with an isocyanate or an isothiocyanate of formula R′₁N═C═Z in which R′₁ and Z are as defined in claim 1, in an inert organic solvent at a temperature comprised between ambient temperature and 60° C.

22. A method for the preparation of a compound of formula (V) 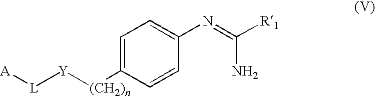
as defined in claim 1 and in which R₁ represents a —N═C(NH₂)R′₁ radical, comprising the step of reacting the aniline of formula (II) 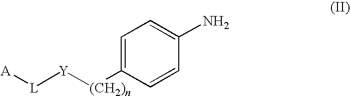
in which A, L, Y and n are as defined in claim 1, with a thioimidate of formula NH═C(SMe)R′₁ in which R′₁ is as defined in claim 1, in a polar solvent, at a temperature comprised between ambient temperature and 80° C. for 2 to 24 hours.

23. A method for the preparation of a compound of formula (VI) 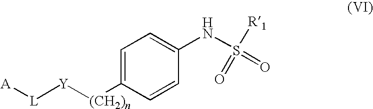
as defined in claim 1 and in which R₁ represents a —NH—S(O)₂—R′₁ radical, comprising the step of reacting the aniline of formula (II) 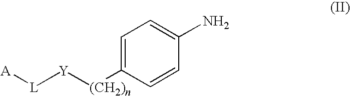
in which A, L, Y and n are as defined in claim 1, with a sulphonyl chloride of formula R′₁S(O)₂Cl in which R′₁ is as defined in claim 1, in an aprotic organic solvent in the presence of a tertiary base, at a temperature of 0 to 60° C. for 1 to 24 hours.

24. A pharmaceutical composition, comprising as active ingredient the compound of formula I of claim 1 or an addition salt with a pharmaceutically acceptable mineral or organic acids of said product of formula I, in combination with and a pharmaceutically acceptable support.

25. A method of treating cell proliferation disorders, comprising administering an effective amount of a compound of claim 1 to a patient in need thereof.

26. A method of treating immune disorders, inflammation, pain, osteoporosis, fibrosis, gastro-intestinal disorders, neurodegencrative diseases including multiple sclerosis and dyskinesia, or Parkinson's disease, comprising administering an effective amount of a compound of claim 1 to a patient in need thereof.

27. The method of claim 25, wherein said cell proliferation disorder is cancer.

28. The compound of claim 1, wherein the compound is an addition salt with a pharmaceutically acceptable mineral or organic acid.

29. The compound of claim 1, wherein the compound is
2-{4-[(cyclobutylcarbonyl)amino]phenyl}ethyl-5,5,8,8-tetramethyl-5,6,7,8-tetrahydronaphthalene-2-carboxylate;
2-(4-nitrophenyl)ethyl-5,5,8,8-tetramethyl-5,6,7,8-tetrahydronaphthalene-2-carboxylate;
2-(4-{[(propylamino)carbonyl]amino}phenyl)ethyl-5,5,8,8-tetramethyl-5,6,7,8-terahydronaphthalene-2-carboxylate;
2-(4-{[(ethylamino)carbonothioyl]amino}phenyl)ethyl-5,5,8,8-tetramethyl-5,6,7,8-tetrahydronaphthalene-2-carboxylate;
4-[(cyclobutylamino)carbonyl]benzyl-5,5,8,8-tetramethyl-5,6,7,8-tetrahydronaphthalene-2-carboxylate;
N-(4-{2-[5-(5,5,8,8-tetramethyl-5,6,7,8-tetrahydronaphthalen-2-yl)-1,3,4-oxadiazol-2-yl]ethyl}phenyl)cyclobutanecarboxamide;
N-allyl-N′-(4-{2-[5-(5,5,8,8-tetramethyl-5,6,7,8-tetrahydronaphthalen-2-yl)-1,3,4-oxadiazol-2-yl]ethyl}phenyl)urea;
N′-(4-{2-[5-(5,5,8,8-tetramethyl-5,6,7,8-tetrahydronaphthalen-2-yl)-1,3,4-oxadiazol-2-yl]ethyl}phenyl)thiophene-2-carboximidamide hydrochloride;
N-[4-({[5-(5,5,8,8-tetramethyl-5,6,7,8-tetrahydronaphthalen-2-yl)-1,3,4-oxadiazol-2-yl]amino}methyl)phenyl]cyclobutanecarboxamide;
N-propyl-N′-[4-([5-(5,5,8,8-tetramethyl-5,6,7,8-tetrahydronaphthalen-2-yl)-1,3,4-oxadiazol-2-yl]amino}methyl)phenyl]urea;
N-(4-{[5-(3,5-di-tert-butylphenyl-1,2,4-oxadiazol-3-yl]methyl}phenyl)cyclobutanecarboxamide;
N-(4-{[5-(3,5-di-tert-butylphenyl)-1,2,4-oxadiazol-3-yl]methyl}phenyl)-N′-propylurea;
N-(4-{2-[4-(5,5,8,8-tetramethyl-5,6,7,8-tetrahydronaphthalen-2-yl)-1,3-oxazol-2-yl]ethyl}phenyl)cyclobutanecarboxamide;
N-(4-{2-[5-(3,5-di-tert-butylphenyl)-1,3,4-thiadiazol-2-yl]ethyl}phenyl)-N′-propylurea;
2-{4-[(methylsulphonyl)amino]phenyl}ethyl-5,5,8,8-tetramethyl-5,6,7,8-tetrahydronaphthalene-2-carboxylate; or
a pharmaceutically acceptable salt thereof.
